(19) United States
(12) Patent Application Publication
(10) Pub. No.: US 2026/0076759 A1
(43) Pub. Date: Mar. 19, 2026

(54) Electronic Torque Measurement Adapter and Self-Check System for a Driving Tool

(71) Applicant: Gauthier Biomedical, Grafton, WI (US)

(72) Inventors: Michael Gauthier, Grafton, WI (US); Austin Braganza, Grafton, WI (US); Paul Seifert, Grafton, WI (US)

(21) Appl. No.: 19/311,556

(22) Filed: Aug. 27, 2025

Related U.S. Application Data
(60) Provisional application No. 63/687,680, filed on Aug. 27, 2024.

(51) Int. Cl. A61B 34/35; A61B 90/00; B25B 23/14; B25B 23/147; G01L 3/10

(52) U.S. Cl. CPC A61B 34/35; B25B 23/141; B25B 23/147; G01L 3/108; A61B 2090/031; A61B 2090/066

(57) ABSTRACT

An electronic torque measuring adapter is provided that can be releasably secured to a driving tool through a drive shaft extending outwardly from one end of the adapter. The adapter has a housing within which is disposed an electronics/control unit capable of measuring torque applied to an implement attached to the adapter opposite the driving tool. The drive shaft is shaped similarly to the engagement end of the fastener-engaging shaft in order to utilize the same connection mechanism within the driving tool to secure the adapter to the driving tool. The adapter can sense and limit the torque applied when a programmed torque limit is reached, which can be stored in the adapter or on a remote platform wirelessly connected to the adapter. Outputs from the adapter can be wirelessly transmitted to the remote platform for recordal of fastener torque values during a medical procedure and generation of associated reports.

### CROSS-REFERENCE TO RELATED APPLICATIONS

This application claims priority from U.S. Provisional Patent Application Ser. No. 63/687,680, filed on Aug. 27, 2024, the entirety of which is expressly incorporated herein by reference for all purposes.

### FIELD OF THE INVENTION

The present invention relates to tools, and more specifically to an adapter connectable to a tool in order to provide electronic measuring and indication of the forces being applied by the tool.

### BACKGROUND OF THE INVENTION

Often, fasteners used to assemble performance critical components are tightened to a specified torque level to introduce a “pretension” in the fastener. As torque is applied to the head of the fastener, beyond a certain level of torque the fastener begins to stretch. This stretch results in the pretension in the fastener which then holds the components together. Accurate and reliable driving tools help ensure the fasteners are tightened to the proper torque specifications.

The tools that are utilized for driving these fasteners vary from simple mechanical types to sophisticated electronic types and include hand-or electrically-operated mechanical drills, wrenches, and other suitable tools, including torque wrenches. There are two common types of mechanical torque wrenches, beam and clicker types. With a beam type torque wrench, a beam bends relative to a non-deflecting beam in response to the torque being applied with the wrench. The amount of deflection of the bending beam relative to the non-deflecting beam indicates the amount of torque applied to the fastener. Clicker type torque wrenches work by preloading a snap mechanism with a spring to release at a specified torque, thereby generating a click noise. Other types of mechanical torque wrenches include indicating, ratcheting, torque limiting, in-line and beam styles of torque wrenches. In an indicating wrench, torque value is measured and displayed on a scale. In a torque limiting wrench, the wrench will drive the fastener until a preset torque value is reached at which point the wrench will slip and cease to transmit the torque applied. In a ratcheting mechanism wrench, in order to drive a fastener into a substrate such as wood or bone it is necessary to rotate the fastener through multiple rotations about its axis. For a hand held tool, in order to drive fasteners, typically the user will have to change their grip or change hands in order to keep driving the fastener due to the limitation of the range of motion of the bones joint in a human hand, which occurs at approximately 100 to 180 degrees depending on the person. A ratcheting mechanism in a fastener driver tool allows the user to rotate the instrument in the opposite direction to the torque being applied without lifting or otherwise disengaging the device driving bit from the fastener and without lifting the hand off the device or changing hands. With a ratcheting mechanism in the tool the user can rotate the tool and drive the fastener through as many degrees of rotation as their hand allows and then ratchet the driving tool in the opposite direction so as to be able to drive the fastener through as many degrees without lifting the hand off the driving tool. Handheld electronic torque wrenches (ETWs) include electronic torque measuring devices and/or mechanisms incorporated directly into the torque wrench (such as any of the above types of torque wrenches), making them more expensive than mechanical torque wrenches, and more accurate as well.

When applying torque to a fastener with an electronic torque wrench, the torque readings indicated on the display device of the electronic torque wrench in a visible manner, such as by a numeric or light indication, and are proportional to the pretension in the fastener due to the applied torque. However, the readings also depend on, among other factors, the under head friction between the head of the fastener and the adjacent surface of the component and the friction between the mating threads. Static friction is greater than dynamic friction. Therefore, when torquing operations are initiated, increased amounts of torque may be required to overcome static friction forces and initiate rotation of the fastener. Therefore, it follows that torque is preferably applied to the fastener in a slow and continuous manner to allow friction forces to stabilize, to help insure accuracy and to help prevent over-torquing, which can result in damage being done to the fastener or the substrate, with extreme cases resulting in destruction of the fastener or substrate. As well, it is often desirable for the user to see both the current torque value (torque being applied at that instant) and the peak torque value (maximum torque applied up to the present instant) simultaneously. However, existing torque wrenches typically display only the current torque value or the peak torque value at any given time.

When a torque wrench is operated in a “tracking mode,” the current torque value is displayed and the user therefore does not necessarily get immediate feedback regarding the actual peak torque value to which the fastener may have been subjected. Although with some electronic torque wrenches it is possible to get this information by downloading the data, this action is typically not instantaneous and, therefore, the operator does not get immediate feedback. On the other hand, when operating in a “peak hold mode,” the display of the electronic torque wrench typically shows only the maximum torque applied to the fastener up to that time. In the peak hold mode, the user is often ignorant of the current torque level, which can lead to either over or under-torquing the fastener.

Another factor that can affect the accuracy of a reading on an electronic torque wrench is the operating temperature.

Strain gages that are used in electronic torque wrenches to measure applied torque are often affected by temperature. Therefore, to obtain accurate torque measurements, it is often necessary to measure the existing temperature and adjust the displayed torque value for a given strain gauge reading.

Another factor that can affect the accuracy of the reading are off-axis loadings while applying torque.

For example, applying a bending load can cause strain that is picked up by the strain gage sensor and reported as a torque value. Strain gages are often designed and positioned in such a way as to reduce the magnitude of off axis loading signals.

Regardless of which type ETW is used, in certain circumstances torque extensions may be required to tighten fasteners that are in locations that the torque wrench will not reach. One of the most common methods of attaching a torque extension to an ETW is to replace the original drive head with an extension that has its own drive head. Articulating joints may also be used. to access hard to reach fasteners.

The power supply required to power a mechanical clutch will increase in proportion with the target torque (torque at which the clutch is designed to slip). When more power is required to power the clutch, a bigger battery is required, this is not desirable since this results in a heavier and more expensive device.

To reduce the power required a gear train may be used to derive mechanical advantage in such a way that even higher target torque limitation mechanisms can be powered with reasonable small battery. Embodiments of gear trains are planetary gear system (epicyclic) and simple (compound) since an idler is involved.

If a gear train or other mechanical leveraging means is in between the ETW and fastener then a different correction factor must be calculated. Typically, the end user calculates a correction factor and either divides or multiplies the desired final actual torque value to be applied to the fastener by this correction factor to determine the final compensated set torque value (as displayed by the ETW) that is to be input into the ETW. Whether the actual torque value is divided by or multiplied by the correction factor is dependent upon the method of determining the correction factor. The final compensated set torque value is the value at which, when displayed, the user ceases to apply torque to the fastener. Typically, the user will only know the final compensated set torque value accurately and is not able to accurately determine the intermediate torque values. In other words, the user only calculates the final compensated set torque value for the set torque and will not be able to continuously monitor the actual torque values during torquing operations as only “compensated” values are displayed by the ETW. This situation can lead to over and under-torquing, possibly resulting in loss of performance of the fasteners.

However, in the prior art these types of electronic torque functions are available only in a dedicated ETW or similar device, with the electronic torque measuring functions disposed within the device as an integral component or components of the ETW. Thus, for any individual wishing to be provided with the increased torque measurement accuracy provided by an ETW, the individual must utilize a dedicated ETW or similar tool or device incorporating the electronic torque measuring functions as a part of the device.

In addition, ETWs indicate torque but do not limit the torque being applied by a person or powered tool motor. In order to limit torque in an ETW, a clutch that would limit the torque being applied by disengaging the transmission when the target torque is reached is required. However, the primary when using an electromagnetic clutch is the amount of power required. With handheld ETWs, the replaceable batteries disposed on the device does not have a sufficient charge to operate the motor and the electromagnetic clutch. As a result, the increased power demand requires a larger, more powerful battery, which can limit the time an individual can hold the driving tool due to the significant increase in weight from the larger battery, or a power cord connection to wall power, which limits the mobility of the driving tool as a result of the connection to the wall and/or the accommodations required for the placement of the cord during a medical procedure.

Thus, it is desirable to develop an adapter for existing driving tool that can be secured to the device in order to enable the adapter to provide enhanced accuracy information regarding the torque applied to a fastener or similar object utilizing the driving tool with the existing power supply for the driving tool.

### SUMMARY OF THE INVENTION

According to one aspect of the present invention, an electronic torque measurement adapter is provided that can be releasably secured to a driving tool. The adapter has a housing within which is disposed an electronics/control unit capable of measuring torque applied through a drive shaft rotatably disposed within the housing and extending outwardly from one end of the housing. The drive shaft, formed with at least one flat surface, is adapted to be inserted within a suitable receptacle, or chuck, in the driving tool that is configured to receive a fastener-engaging shaft, also formed with at least one flat surface. The drive shaft is shaped similarly to the engagement end of the fastener-engaging shaft in order to utilize the same connection mechanism within the driving tool to secure the drive shaft and adapter to the driving tool.

Opposite the drive shaft, the housing includes a receptacle for receiving a fastener-engaging shaft. The receptacle includes an engagement mechanism capable of securely holding the fastener-engaging shaft within the housing and operably connected to the drive shaft. In this manner the adapter enables the rotation of the drive shaft performed by the driving tool to be transmitted through the adapter to the fastener-engaging shaft in order to drive a fastener engaged by the fastener-engaging shaft opposite the adapter.

In addition, as the drive shaft rotates the control unit can determine the torque applied to the fastener and record, transmit and/or display the torque to the individual utilizing the driving tool.

The adapter can utilize an internal power supply for the operation of the control unit, or can include connections for engagement with the driving tool to enable a power supply for the driving tool to power the control unit within the adapter.

Further, the adapter can include a clutch mechanism operably connected to the electronics/control unit to actively control the torque output by the driving tool. The power source for the mechanism can be provided by the internal power source for the adapter or from the power source for the driving tool.

According to still another aspect of the present invention, an electronic torque measuring adapter is provided with torque adjustment capability, display of desired torque settings, actual torque achieved display, torque measuring capability and optionally a shutoff at a predetermined torque level. The adapter provides repeatable and accurate torque application without regard to operator capability within a sterile operating environment.

Among the several features, objects and advantages of the present disclosure are the provision of an electronic torque measurement adapter:
measures applied torque precisely by electronically sensing torque during operation;
allows convenient operator selection of the predetermined torque level and displays the predetermined torque level;
does not require external sensing and control circuits but are instead integrated completely into the tool in a manner that enables the wrench to be utilized in a sterile environment;
provides for torque sensing and torque control by electronic feedback;
does not require an external source of electrical power but is instead capable of operating solely under battery power;
can include a gear train to provide mechanical advantage and higher torque with a given power source;
can include a powered driver system that is battery powered or wall powered; can be integrated into typical configurations of existing driving tools by use of sensing and control elements which will not interfere with normal speed and convenience of operation of the driving tool;
uses a torque-sensing technology of such significant economy and relative simplicity as to make possible portable digital torque-responsive, and optionally torque-controlling wrenches; and
provides such torque-responsive, and optionally torque-controlling tools with such economy and simplicity as to be suited for medical use in sterile environments, as well as use in many other technical areas.

According to still a further aspect of the present disclosure, an electronic torque measuring adapter, system, and method of using the adapter and system is provided for tightening and standardizing the forces associated with a fastener system for medical procedures. In one embodiment, the system includes access to a database of fastener configuration information for various medical procedures, specifically as they relate to the particular individual on which the procedure is to be performed. Information is provided from the database to the adapter apparatus. The adapter provides verification of the information and verification of application of the information by the use of the driving tool to which the adapter is connected. After use, the adapter transfers the information back to the system to provide a historical record of the procedure and the torque values applied by the physician using the driving tool to which the adapter is connected to the respective fasteners.

According to still another aspect of the present disclosure, the electronic torque measuring adapter includes a coupling device or coupler and the adapter. The coupling device receives information from the system and transfers it to the adapter. Once the fastener configuration information is received, the adapter is removed from the coupler and is used to establish torque settings for use in the fastener torque process. Verification of the tightening process is recorded at the adapter during use and transmitted back to the coupler. The coupler then transfers the information to the system.

According to still a further aspect of the present disclosure, the electronic torque measurement adapter and system includes a driving device management server which communicates with the control unit/microprocessor in the adapter. The adapter is connected to the server to collect information about the procedure/subject from the server. The device management server then delivers corresponding fastener configuration information to the coupler for transfer to the electronic torque measurement adapter. The adapter utilizes the information in the fastener tightening process to alert the physician of the proper torque value for the particular fastener being tightened in the procedure. Verification of the information can be recorded at and/or by the adapter and transferred back to the coupler when the adapter is placed in/engaged with the coupler. Information transferred to the coupler can be transmitted to the procedure management server for verification, transaction completion and storage.

According to still another aspect of the present disclosure, the data regarding the use of the adapter and driving tool in performing the fastener tightening procedure or process stored in the control unit/microprocessor can be transferred to the coupler and/or server to record the usage of the driving tool. This data can be stored in the server for use in determining the necessary calibration of the adapter, based on various parameters such as the number of uses of the adapter, and the overall length of time of use of the adapter, among others.

According to still another aspect of the present disclosure, the adapter can be designed to be pre-calibrated for accurate determination of the torque applied to a fastener in a single use, such that the adapter can be disposed of after use in a single procedure or process.

According to still a further aspect of the present disclosure a companion system or mobile application/ETW Software Application (App) employed in conjunction with the torque-application device or tool or adapter is capable of:
displaying real-time, torque data as the surgeon is applying torque to the fastener in surgery.
applying a torque type tag and location tag to the recordings. The user can export all the recordings in a report organized using the tags providing clear indication that all fasteners were tightened and to the appropriate torque.
providing verification of whether or not all fasteners were tightened as intended.
generation of data/logs regarding the use of the tool for review and analysis post operatively.
implant (e.g., pedicle screw, rod, set screw, etc.) identification information can be entered into /ta/ logs, such as by scanning a barcode or RFID tags with a mobile device on which the companion system or ETW Software Application is operating and/or directly into the companion system or ETW Software Application.
in a self-check mode of operation, the companion system or ETW Software Application has the ability to check if the ETW adapter device is working within design parameters/ready for use, e.g., is properly calibrated.

Other aspects, features and advantages of the present invention will be set forth in part in the description which follows and the accompanying drawings, wherein the embodiments of the disclosure are described and shown, and in part will become apparent upon examination of the following detailed description taken in conjunction with the accompanying drawings.

### BRIEF DESCRIPTION OF THE DRAWINGS

The drawings illustrate the best mode currently contemplated of practicing the present invention.

In the drawings:

FIG. 1 is an isometric view of a first embodiment of an electronic driving tool and electronic torque measurement adapter constructed according to the present disclosure;

FIG. 2 is an isometric view of the adapter of FIG. 1 attached to a fastener-engaging shaft according to the present disclosure;

FIGS. 3A-3B are exploded views of the adapter and driving tool of FIG. 1 with a gear assembly according to the present disclosure;

FIG. 4 is an isometric view of a clutch mechanism of the adapter of FIG. 3A according to the present disclosure;

FIGS. 5A-5D are exploded isometric views of the clutch mechanism of FIG. 4 according to the present disclosure;

FIG. 6 is an isometric view of a driven implement operably connected to the adapter and electronic driving tool constructed according to the present disclosure;

FIG. 7 is an isometric view of the adapter of FIG. 1 according to the present disclosure;

FIG. 8 is a cross-sectional view along line 8-8 of FIG. 7 according to the present disclosure;

FIG. 9 is a side elevation view of the cross-sectional view of FIG. 7 according to the present disclosure;

FIG. 10-10B are view of alignment surfaces on a shaft on or engaged with the adapter of FIG. 7 according to the present disclosure;

FIG. 11 is an exploded, isometric view of driven implement utilized with the adapter of FIG. 10 according to the present disclosure;

FIG. 12 is a cross-sectional view of the adapter of FIG. 7 showing a fastener-engaging shaft of FIG. 10 inserted within the adapter according to the present disclosure;

FIG. 13 is an isometric view of a third embodiment of the adapter including an electrical connection ring constructed according to the present disclosure;

FIG. 14 is an isometric view of a fourth embodiment of the adapter including a connection collar constructed according to the present disclosure;

FIG. 15 is an isometric view of a fifth embodiment including an adapter integrated with an in-line torque wrench constructed according to the present disclosure.

FIG. 16 is an isometric view of a sixth embodiment including an adapter releasably connected to an in-line torque wrench constructed according to the present disclosure.

FIG. 17 is an isometric view of a seventh embodiment including an adapter integrated with a T-handle torque wrench constructed according to the present disclosure.

FIG. 18 is an isometric view of an eighth embodiment including an adapter releasably connected to a T-handle torque wrench constructed according to the present disclosure.

FIGS. 19-19C are various views of a cover for the adapter of FIG. 7 constructed according to the present disclosure.

FIG. 20 is an isometric view of a gripping ring of the adapter of FIG. 7 constructed according to the present disclosure.

FIG. 21 is a schematic view of a torque data recording system constructed according to the present disclosure.

FIGS. 22A-22B are schematic views of a sensing device including torque sensors disposed thereon constructed according to the present disclosure.

FIG. 23 is a schematic view of a torque sensor constructed according to the present disclosure.

FIG. 24 is a schematic view of a display presented by the torque data recording and analysis system on a device containing the system according to the present disclosure.

FIG. 25 is a schematic view of another embodiment of a torque data recording and analysis system constructed according to the present disclosure.

FIGS. 26A-26C illustrate representations of the display of a remote device or platform in the memory of which is contained executable instructions for the torque data recording and analysis system in various initialization steps of the method of use of the system according to the present disclosure.

FIG. 27 illustrates representations of the display of a remote device or platform in the memory of which is contained executable instructions for the torque data recording and analysis system in various detection steps of the method of use of the system according to the present disclosure.

FIG. 28 illustrates representations of the display of a remote device or platform in the memory of which is contained executable instructions for the torque data recording and analysis system in various selection steps of the method of use of the system according to the present disclosure.

FIG. 29 illustrates representations of the display of a remote device or platform in the memory of which is contained executable instructions for the torque data recording and analysis system in various further selection steps of the method of use of the system according to the present disclosure.

FIG. 30 illustrates representations of the display of a remote device or platform in the memory of which is contained executable instructions for the torque data recording and analysis system in various recording steps of the method of use of the system according to the present disclosure.

FIGS. 31A-31C illustrate representations of the display of a remote device or platform in the memory of which is contained executable instructions for the torque data recording and analysis system in various record access steps of the method of use of the system according to the present disclosure.

FIG. 32 illustrates another embodiment of a representation of the display of a remote device or platform in the memory of which is contained executable instructions for the torque data recording and analysis system in a recording step of the method of use of the system according to the present disclosure.

FIG. 33 illustrates another embodiment of a representation of the display of a remote device or platform in the memory of which is contained executable instructions for the torque data recording system in a record access step of the method of use of the system according to the present disclosure.

FIG. 34 is an illustration of an exemplary embodiment of a report generated on a display of the remote device or platform in the memory of which is contained executable instructions for the torque data recording and analysis system for a recorded torque event according to the present disclosure.

FIG. 35 is a schematic view of an operating theater in which the torque data recording and analysis system is employed on a remote device or platform according to the present disclosure.

FIG. 36 a schematic view of an operating theater in the torque data recording and analysis system is employed on a remote device or platform in conjunction with a surgical robot according to the present disclosure.

FIGS. 37-37B are partially broken away, isometric views of the engagement of the cover with the gripping ring of FIG. 20 according to the present disclosure.

FIGS. 38-38G are various views illustrating the manner of engagement of the arms and tabs on the gripping ring with an implement inserted within the adapter of FIG. 7 according to the present disclosure.

FIGS. 39-39F are various views of the rotational positions of the cover relative to the gripping ring and adapter to selectively lock an implement within the adapter and to selectively turn on power to the adapter according to the present disclosure.

FIGS. 40-40A are perspective view of the use of tags on the fasteners or other medical devices used in the procedure employing the adapter and tool of FIGS. 1 and 7, and which tags can be scanned or read by the torque data recording and analysis system for storage in the stored record and report for the procedure.

FIG. 41 shows view of the various steps and components for the connection of a pedicle screw and rod in an exemplary spinal procedure employing the adapter and tool of FIGS. 1 and 7.

FIGS. 42-42A illustrate the components of the fastener and the implements engaged by the adapter and tool of FIGS. 1 and 7 in performing an exemplary spinal procedure.

FIG. 43 is a perspective view of the adapter and tool of FIGS. 1 and 7 in sterilized and unsterilized configurations for use in an exemplary spinal procedure.

FIG. 44 is a diagram showing interaction(s) between the adapter and tool of FIGS. 1 and 7, the remote device or platform in the memory of which is contained executable instructions for the torque data recording and analysis system and other interconnected elements including the user.

FIG. 45 is a schematic view of an exemplary embodiment of the hardware of the adapter and tool of FIGS. 1 and 7, the remote device or platform in the memory of which is contained executable instructions for the torque data recording and analysis system.

### DETAILED DESCRIPTION OF THE INVENTION

Reference will now be made in detail to various embodiments of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents.

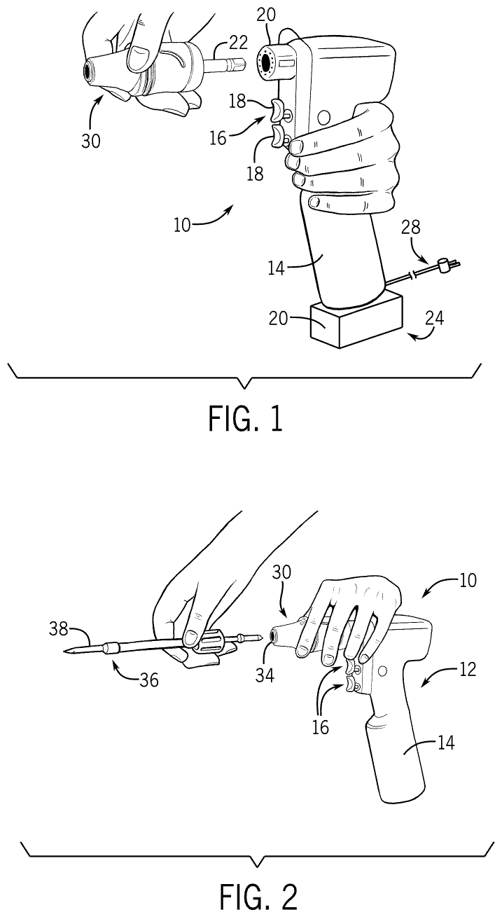

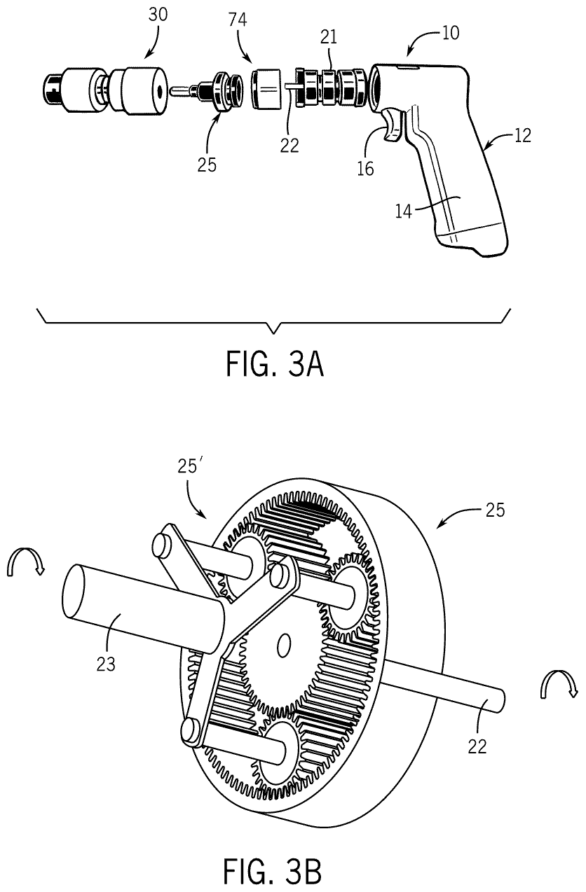

Referring now to FIGS. 1-3B, a driving tool 10, such as, but not limited to a drill 11, includes a body 12 from which extends a handle 14. The body 12 also includes a number of control elements 16, such as switches 18, and a sleeve 20 for receiving a shaft 22 therein. The sleeve 20 is operably connected to a motor 21 that is disposed within the body 12 and is operable to rotate the sleeve 20 and the shaft 22 engaged within the sleeve 20 under the control of the control elements 16 operably connected to and able to selectively operate the motor. The body 12 can additionally include an internal power source (not shown), such as a rechargeable battery, or an external power source 24, such as a battery 26 and/or a power cord 28 disposed on or connectable to the handle 14 and operably connected to the motor 21 to drive/rotate a motor output shaft 23 connected to a gear assembly 25 that functions to convert the speed of the output shaft 23, e.g., operates to reduce or increase the rotational speed, as desired for rotation of a device and/or shaft 22 engaged with the assembly 25. The gear assembly 25 can be formed as desired, but in the illustrated exemplary embodiment of FIG. 3B the gear assembly 25 is formed as a planetary gear assembly 25′.

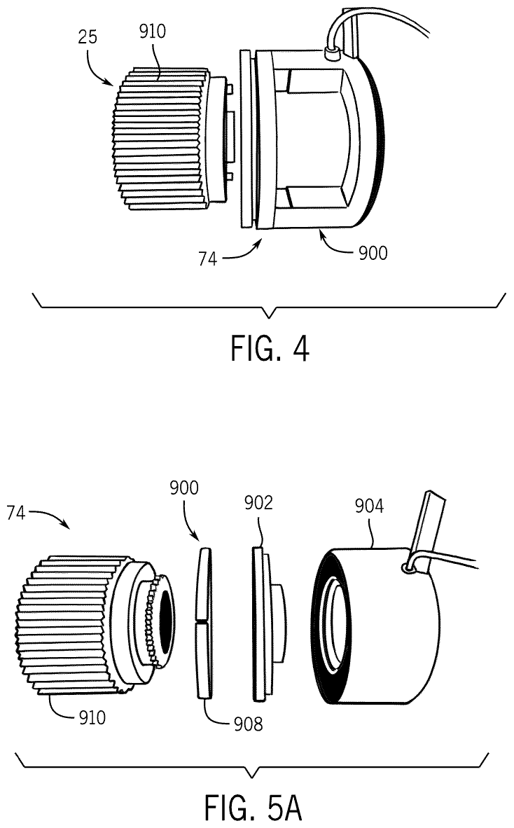

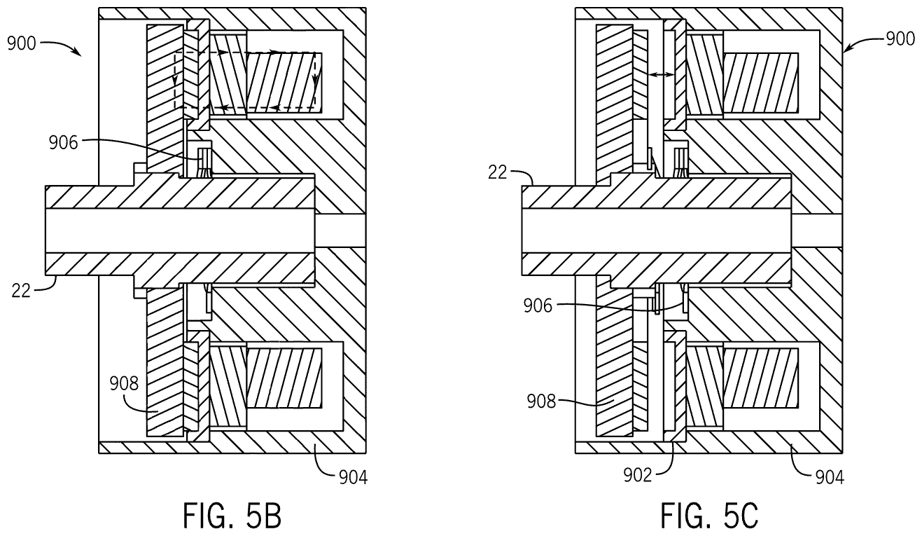

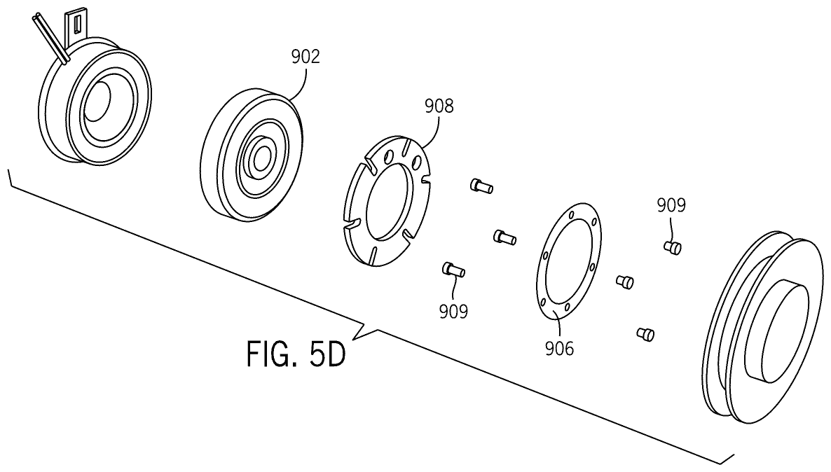

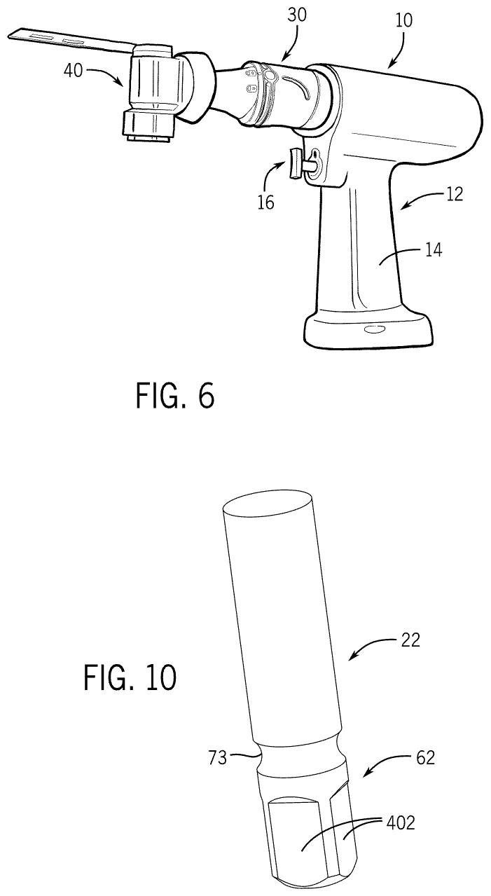

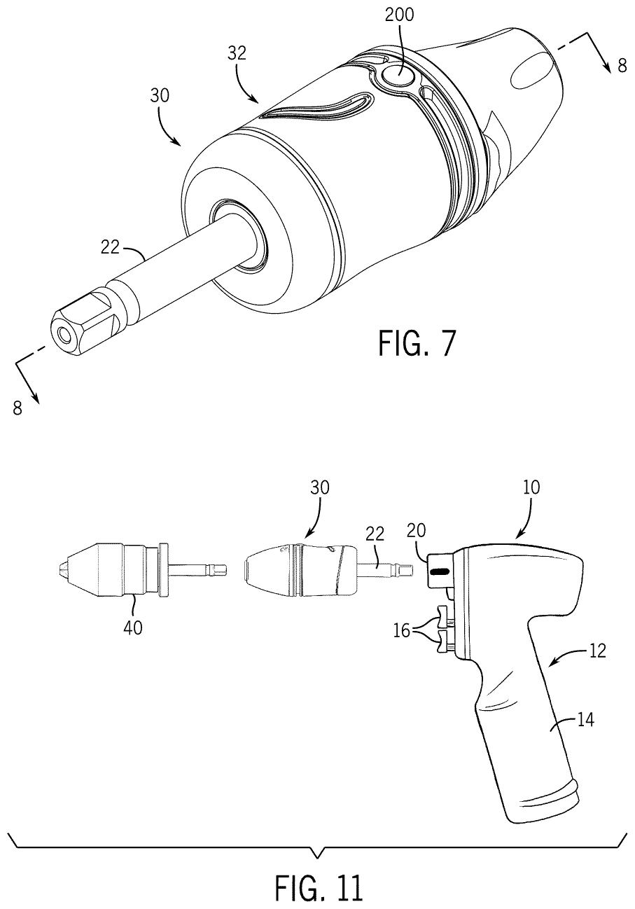

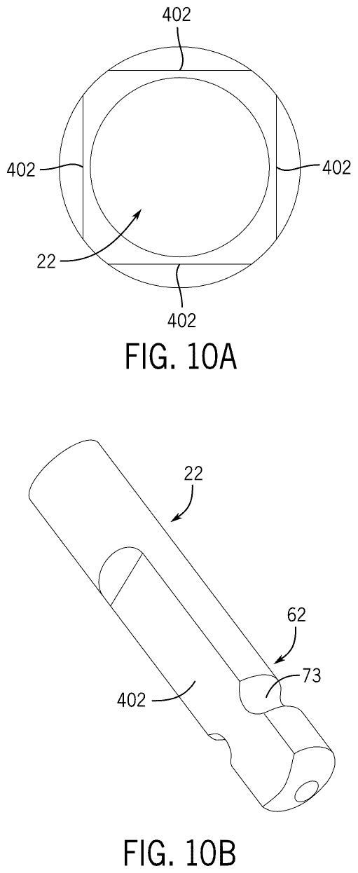

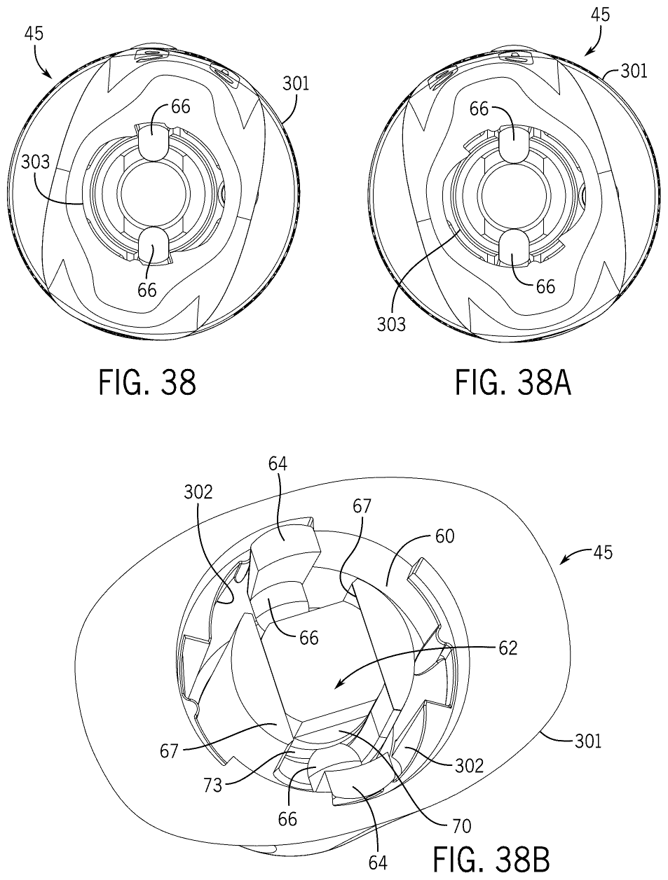

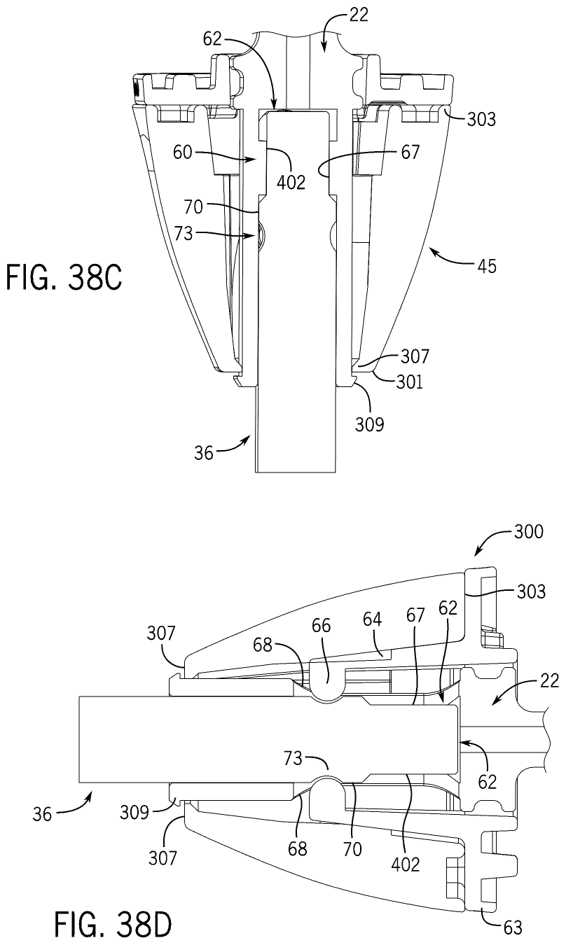

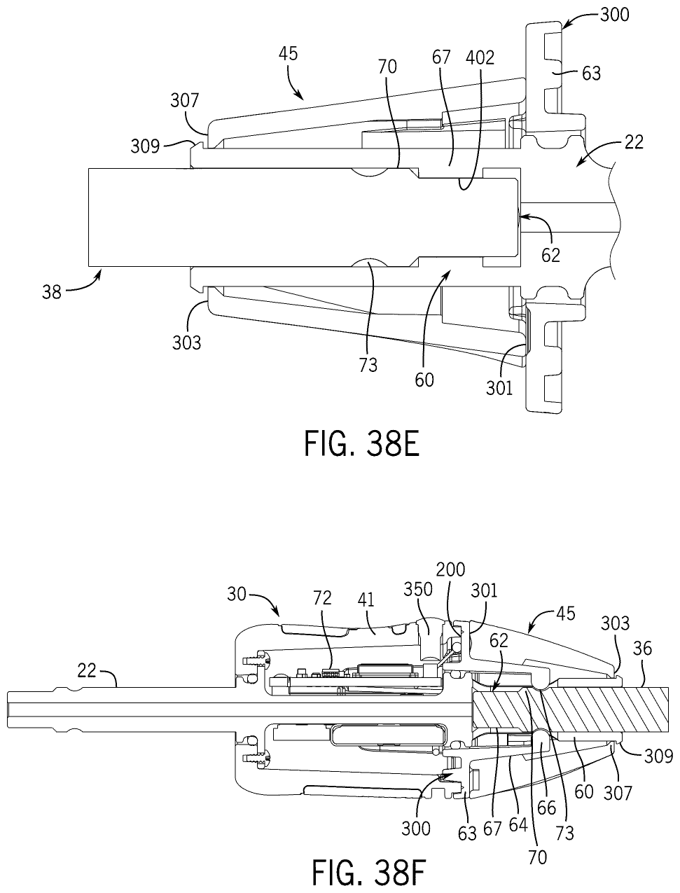

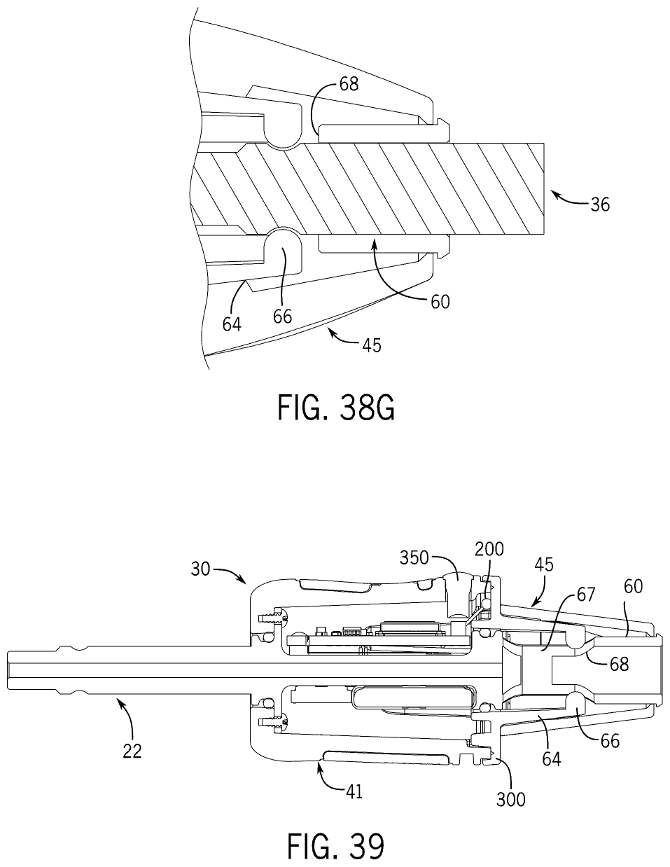

Referring now to FIGS. 7-12, an adapter 30 is releasably connected to the tool 10 and includes the shaft 22. The shaft 22 extends outwardly from a body 32 of the adapter 30, which includes a receptacle 34 disposed within the body 30 opposite the shaft 22 affixed to the shaft 22 for rotation therewith. The receptacle 34 is configured to receive an implement 36 therein, such as a fastener-engaging shaft 38 (FIG. 2) or a driven implement 40 (FIG. 6) therein. With reference now to each of FIGS. 10-10B, both the shaft 22 and the implement 36/fastener-engaging shaft 38 each include an end 400 having a number of flat surfaces 402, with four (4) flat surfaces 402 shown in the illustrated exemplary embodiment of FIGS. 10 and 10A, and one (1) flat surface 402 in the exemplary embodiment of FIG. 10B, formed thereon in any suitable configuration. The flat surfaces 402 enable the alignment of the shaft 22 within the tool 10 and the implement 36/fastener-engaging shaft 38 within the receptacle 34 of the adapter 30 or other aperture(s) receiving the shaft 22 or the implement 36/fastener-engaging shaft 38 for proper alignment and secure engagement therein (FIG. 38B). The flat surfaces 402 also enable the shaft 22/adapter 30 to rotate in response to rotation of the motor output shaft 23 of the tool 10, and the implement 36/fastener-engaging shaft 38 to rotate in conjunction with the adapter 30. Concurrently, the flat surfaces 402 also enable transmission of torque from the implement 36/fastener-engaging shaft 38 to the adapter 30 for measurement by the adapter 30, and from the shaft 22/adapter 30 to the tool 10 to control the operation of any clutch mechanism 74 (FIGS. 4-5D) disposed within the adapter 30 and/or tool 10.

The shaft 22 extends through an aperture 42 into the interior 47 of the body 32, which includes a housing 41 including the aperture 42 at one end and an open end 43 opposite the aperture 42, and a cover 45 secured over the open end 43. The shaft 22 includes an outer ring 44 that is disposed within the aperture 42 and has a diameter similar to the diameter of the aperture 42 and an inner ring 46 that is located within the interior 47 and has a diameter greater than the aperture 42. The radially outwardly extending outer ring 44 and inner ring 46 define a groove 48 therebetween in which is disposed a seal 50 that engages the body 32 within the aperture 42 to seal off the interior 47 from the exterior of the body 32. Further, the inner ring 46 overlaps a portion of the housing 41 around the aperture 42 and is secured thereto by fasteners 55 engaged between the inner ring 44 and the housing 41 to fix the shaft 22 to the housing 41/body 32.

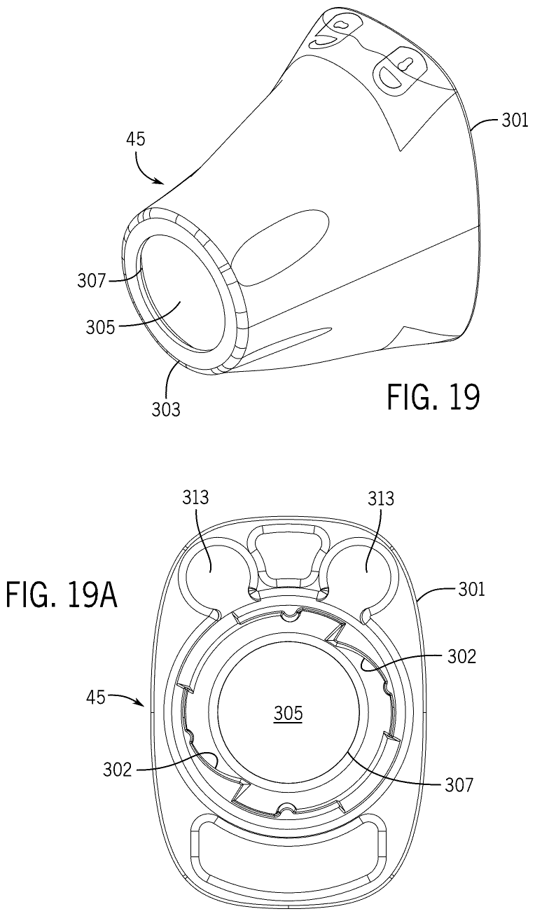

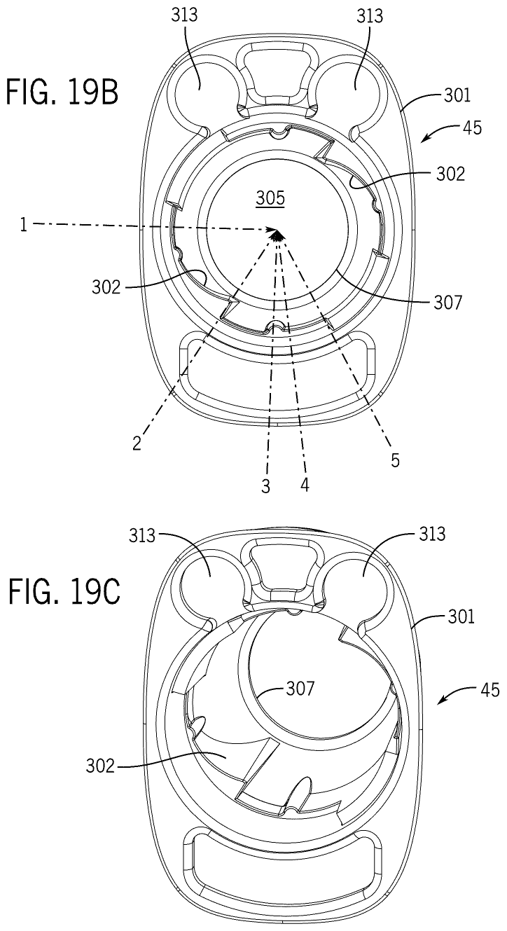

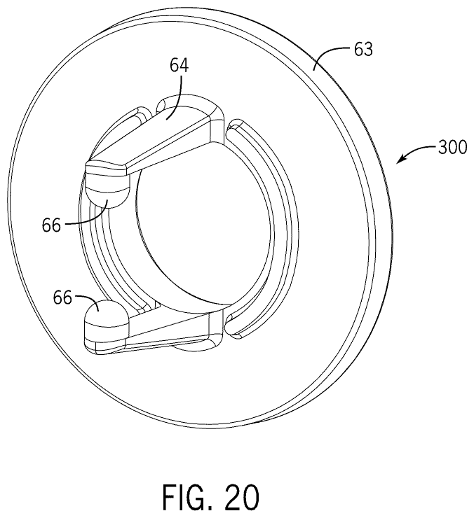

The shaft 22 extends through the interior 47 and includes a collar 52 disposed within and in alignment with an opening 54 formed in the cover 45, as shown in the exemplary embodiment illustrated in FIGS. 19-19C. The collar includes a central groove 56 within which is disposed a seal 58 engaged between the collar 52 and the interior of the opening 54 in the cover 45 to seal the opening 54.

In the embodiment of FIG. 9, a sleeve 60, which defines the receptacle 34 secured to the shaft 22 within the housing 41 where sleeve 60 can be fixed to or integrally formed with the shaft 22, extends outwardly from the collar 52 and is shaped to receive and engage the engagement end 62 of the implement 36 to hold the implement 36 on the adapter 30. The sleeve 60 is disposed within an enclosure 64 located around the sleeve 60 and including a mechanism capable of securing, e.g., frictionally securing, the engagement end 62 within the sleeve 60, such as a friction chuck mechanism, among other suitable mechanisms.

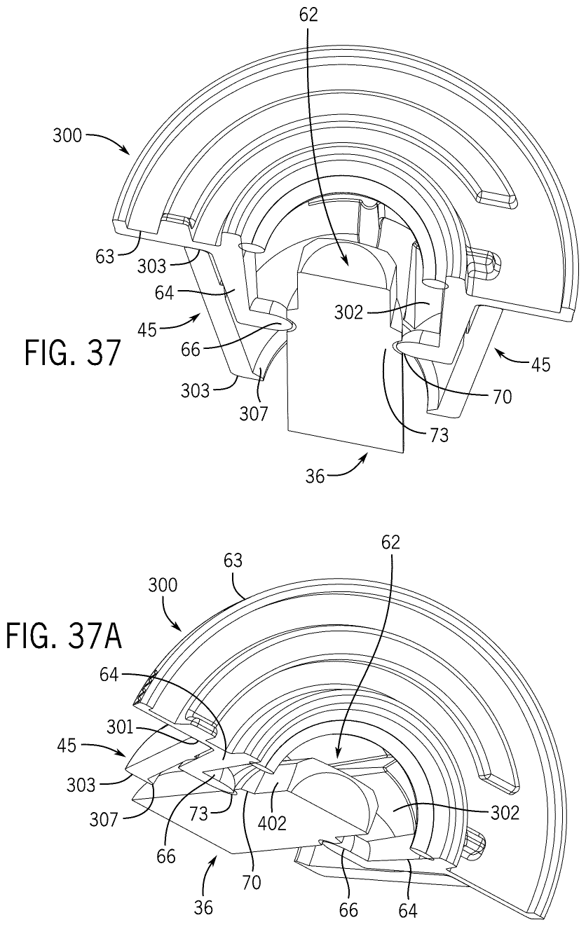

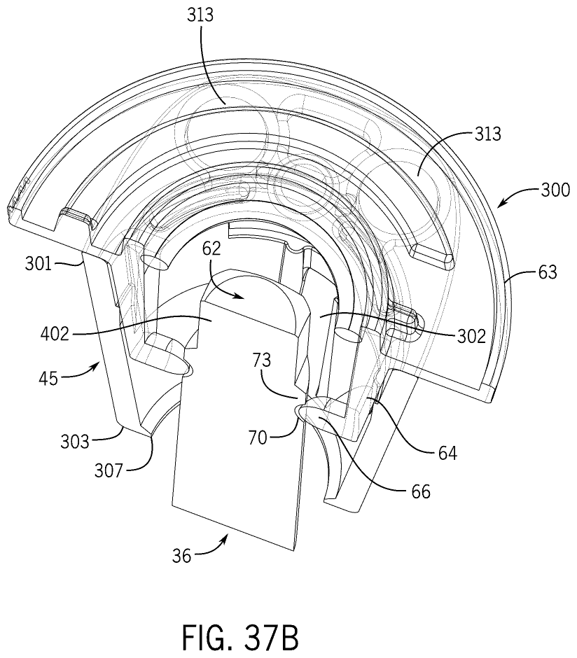

In the embodiment of FIGS. 7, 8, 12, 19-20 and 37-38G, as an alternative to or to be used in conjunction with the chuck mechanism of FIG. 9, a chuck mechanism or gripping ring 300 is disposed on the adapter 30 between the sleeve 60 and the cover 45. In one exemplary embodiment, the gripping ring 300 is engaged, e.g., sealed in a fluid-tight manner, to the adapter 30 around the sleeve 60, e.g., to the housing 41, to enable the cover 45 to rotate relative to the ring 300. The ring 300 includes a base 63 secured to the housing 41 and pair of opposed arms 64 extending outwardly from the base 63 in alignment with the sleeve 60, which is formed separately from but secured to the collar 52. The arms 64 terminate in tabs 66 that extend radially inwardly through openings 68 in the sleeve 60. The arms 64 are flexible, but are biased inwardly towards the sleeve 60 retain the tabs 66 within the openings 68 in the sleeve 60. The tabs 66 can be engaged within a locking ridge 70 disposed on the implement 36 when the implement 36 is inserted within the sleeve 60 to at least partially displace the tabs 66 out of the sleeve 60 through the openings 68 and allow the locking ridge 70 to pass the tabs 66, where the flexibility of the arms 64 reinserts the tabs 66 through the openings 68 to engage a channel or groove 73 on the implement 36 adjacent the ridge 70 and hold the implement 36 within the sleeve 60.

To engage the tabs 66 with the shaft/implement 36, the cover 45 can be rotated to press the tabs 66 towards the implement 36, as best shown in FIGS. 12 and 37-38G, the selectively lock and unlock the implement 36 within the cover 45. The cover 45 includes a wide end 301 disposed adjacent the housing 41 and a narrow end 303 opposite the wide end 303. The cover 45 defines a channel 305 within which is positioned the arms 62 of the gripping ring 300 and the sleeve 60. The channel 305 includes a radially-inwardly extending securing ring 307 at the end of the channel 305 near the narrow end 303 which engages a circumferential projection 309 on the sleeve 60 to hold the cover 45 around the sleeve 60 and the securing ring 300 in a rotatable manner.

To perform the locking function with the gripping ring 300, the cover 45 includes a number of engaging surfaces 302 on the interior of the cover 45 that can selectively engage and move or deflect the arms 64 to position the tabs 66 out of or within the sleeve 60 in engagement with the implement 36. Further, the sleeve 60 includes an alignment surface 67 can be aligned with at least one flat panel 71 or surface 402 on the implement 36. The alignment surface 67 properly locates the implement 36 within the cover 45 and sleeve 60 to allow engagement of the tabs 66 with the groove 73 and to enable effective transfer of the torque from the implement 36 to the cover 45 and adapter 30, such as for measurement by the adapter 30.

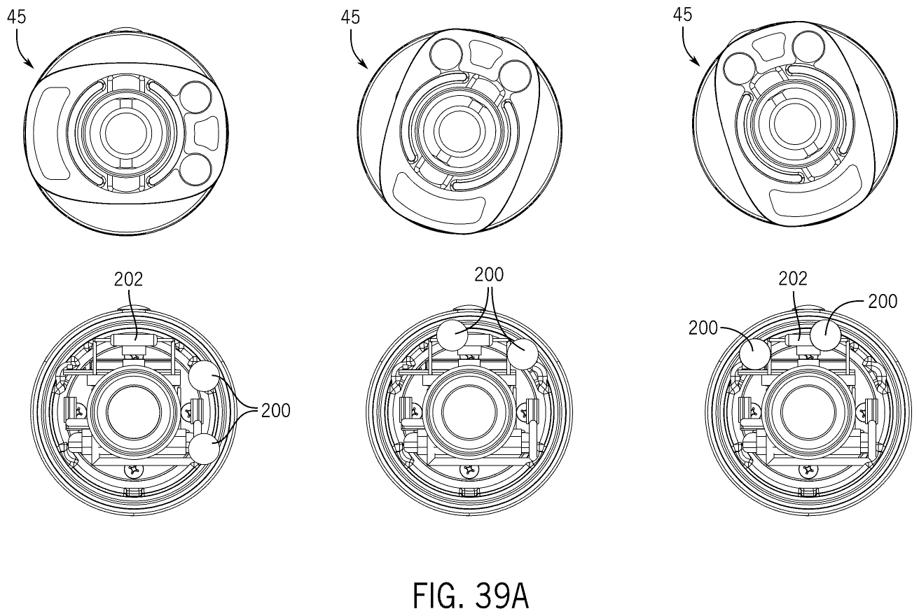

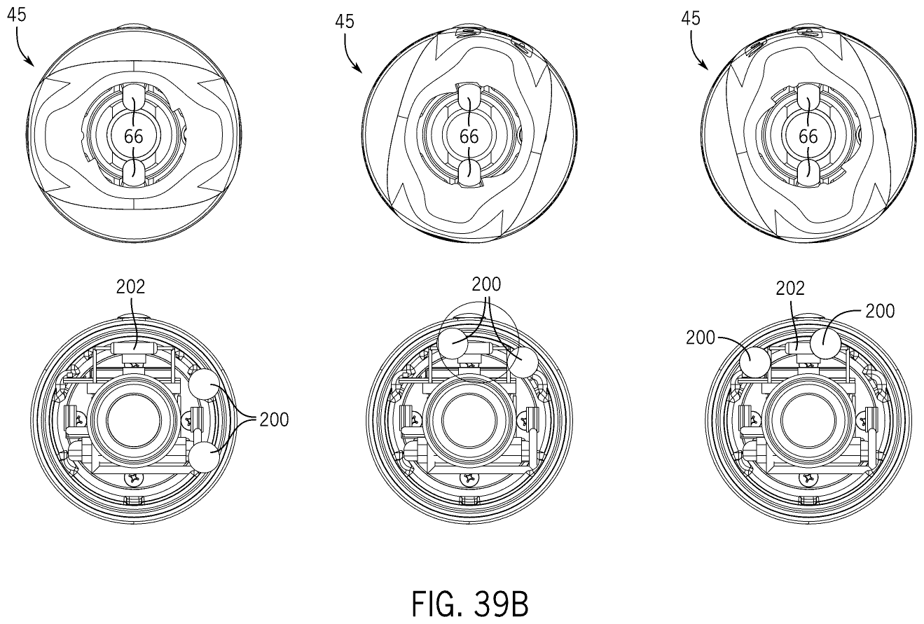

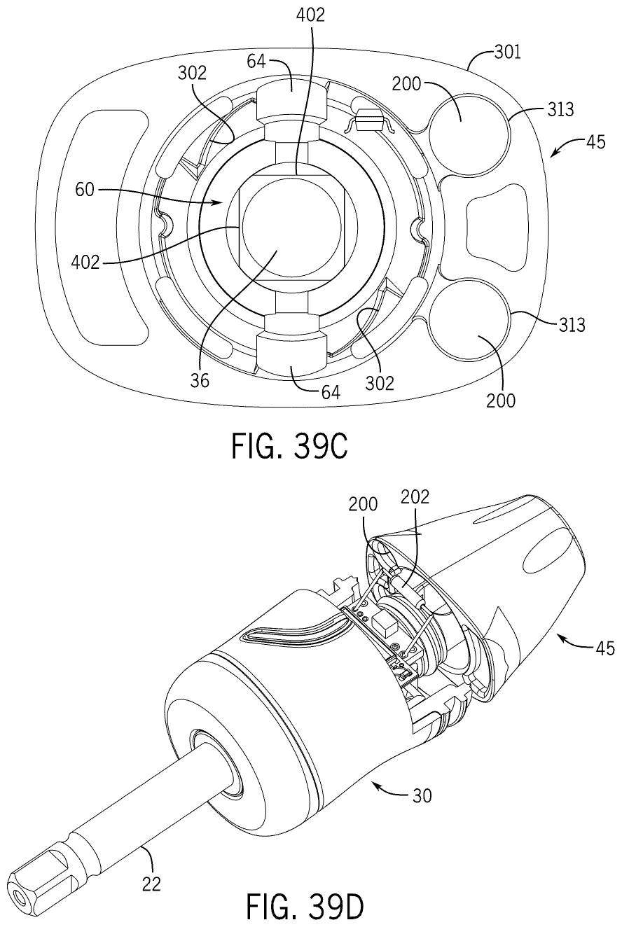

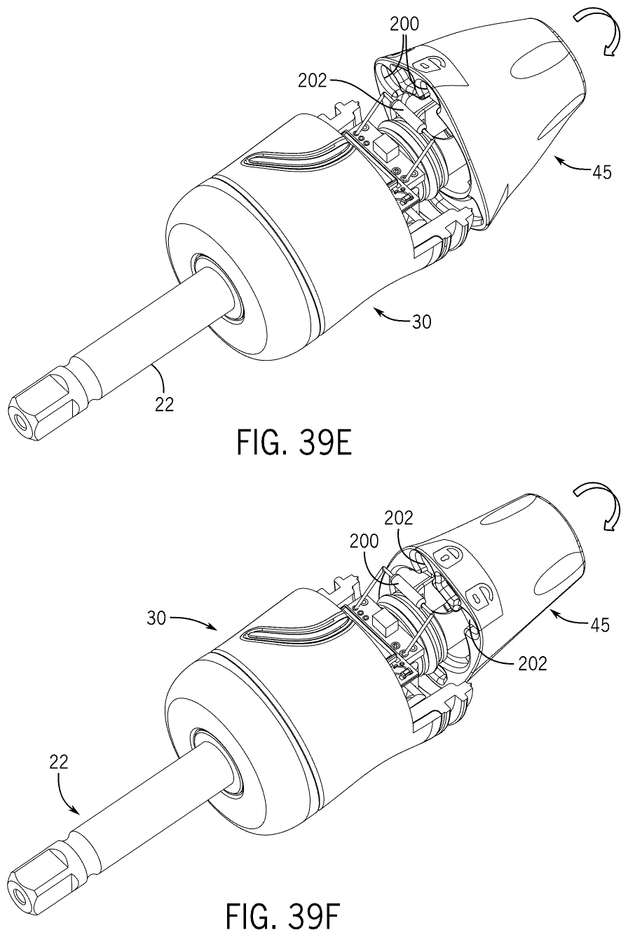

In addition, referring to FIGS. 39-39F, in which a portion of the adapter 30 is removed for visualization of the interior of the adapter 30, the cover 45 includes a pair of magnets 200 located in recesses 313 formed in the cover 45. The magnets 200 can be moved into positions adjacent a switch 202, such as a reed switch 204, in order to supply power to the adapter 30. In one exemplary embodiment, in a first position shown in FIG. 39D, the cover 45 is in a first position relative to the remainder of the adapter 30 where the magnets 200 are both displaced from the switch 202, such that the adapter 30 in not powered, either by an internal battery (in the adapter 30 or tool 10) or a wired supply connected to the tool 10 and operably connected to the adapter 30. In a second position shown in FIG. 39E, the cover 45 is rotated to position one of the magnets 200 in at least partial alignment with the switch 202, to provide power to the adapter 30. In this position, with the configuration of the cover 45 and the arms 64 and tabs 66, the second position can leave the tabs 66 able to receive the implement 36 within the cover 45. In a third position shown in FIG. 39F, the cover 45 is further rotated to position the other of the magnets 200 in at least partial alignment with the switch, maintaining power to the adapter 30, but also pressing the tabs 66 into engagement with the implement 36 inserted within the cover 45 using the engaging surfaces 302 to lock the implement 36 in engagement with the adapter 30 for use of the implement 36 with the adapter 30 and tool 10.

The interior 47 of the housing 41 also encloses a printed circuit board (PCB)/electronics/control unit 72 operably connected to the switch 202 and capable of determining/analyzing the amount of torque applied to a fastener engaged by the implement 36 that is connected to the adapter 30. The electronics/control unit 72 can provide various indications to the user of the sensed or measured levels, amount and/or proximity of the applied torque to a preset value stored in a suitable electronic memory (not shown) disposed within the adapter 30 and operably connected to the control unit 72 in any number of manners, including but not limited to signals, temperature, orientation, vibrations, side loads and torque bending loads using a user interface that can include one or more of lights, colors, sound alarm, and vibration among other suitable notification types and processes.

As best shown in the exemplary embodiments of FIGS. 4-5D, the adapter 30 can optionally additionally include a clutch mechanism 74 located within the housing 41 and selectively operable by the control unit 72 within the adapter 30. The clutch mechanism 74 is operably connected to the control unit 72, and in the illustrated exemplary embodiment is an electromagnetic clutch mechanism 900 including a rotor 902 operably connected to the motor 21 for rotation in response to the operation of the motor 21. A selectively energizable field coil 904 (operably connected to power source 24) is disposed adjacent the rotor 902 and includes a spring 906 thereon. An armature 908 is disposed on/fixed to an output gear 910, which can form part of the gear assembly 25 described previously, and is engaged by the spring 906, where rotor 902, spring 906, and armature 908 are alignable with suitable fasteners 909. In operation, as best illustrated in FIGS. 5B and 5C, when a current is applied to the field coil 904, the electromagnetic field generated by the field coil 904 attracts the armature 908 and output gear 910 towards the rotor 902 against the bias of the spring 906 (FIG. 5B). In this configuration the armature 908 is directly engaged by the rotor 902 and can rotate in conjunction with the rotor 902 via the operation of the motor 21. Alternatively, when the field coil 904 is de-energized, the force of the spring 906 displaces the armature 908 and output gear 910 from the rotor 902, such that the rotation of the rotor 902 is not transmitted to the output gear 910 and gear assembly 25. Based on torque measurements obtained by the control unit 72, the control unit 72 can selectively operate the clutch mechanism 74 to slow or stop the rotation of the shaft 22, and thus the adapter 30, in order to prevent over torquing of the fastener (not shown) by the tool 10, particularly in relation to any stored preset torque value associated with the fastener(s).

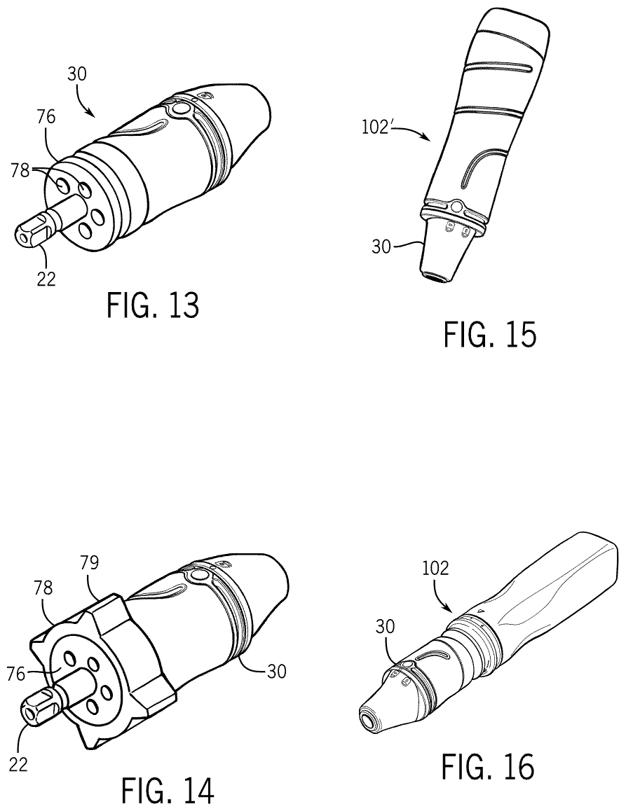

Referring now to FIGS. 13 and 14, either as a substitute for or for use in conjunction with the switch 202 and magnets 200, in order to provide a data transfer connection and supply power to/from the electronics/control unit 72 and the electromagnetic clutch 74, if present, the adapter 30 includes an electric connection ring 76, disposed around the shaft 22. The ring 76 includes a number of ports 78 that are adapted to receive and engage complementary shaped plugs (not shown) disposed on the body 12 and to which are operably connected the power source 24, PCB/electronics/control unit 72 and data transfer devices (not shown) within the body 12. In this manner, the power and/or data transfer functionality for the adapter 30 can be provided by the body 12. The ring 76 can be formed with a collar 79 disposed around the ring 76 to aid in the connection of the ring 76 to the appropriate plugs on the body 12 for desired functionality of the adapter 30. The signal connection lines between the tool 10 and the ports 78 connect the adapter 30 and electronics/control unit 30 to the powered tool 10 to use its larger capacity, powerful battery 26 or power source 24. This exemplary embodiment for the adapter 30 eliminates the need to add a separate battery for the adapter 30, though use of a rechargeable and/or separable battery/magnet 200 on the adapter 30 is also contemplated as being within the scope of the present disclosure, as described previously. Further, this exemplary design eliminates the need to add a separate battery pack and therefore weight and size to the front of the tool where the adapter 30 is typically situated, and can use existing the surgical power tool battery pack and power cord. There are improved ergonomics with the battery pack situated on the power tool 10 above the gripped portion of the handle or below it.

The adapter 30 can also be configured for use in a robotic environment or configuration, where the adapter 30 can be affixed to an arm 811 of the robot 810 (FIG. 36) performing the procedure, to provide the measurement of the torque being applied. This or any other embodiment may also include accelerometers, gyroscopic devices, spatial sensors, e.g., time-of-flight sensors, LIDAR sensors/systems, and/or navigation orbs (not shown) or other similar devices disposed on the adapter 30 in order to locate the adapter 30 and any associated structures, such as the tool 10 or a robotic arm 811, in an operating theater using know navigation technology.

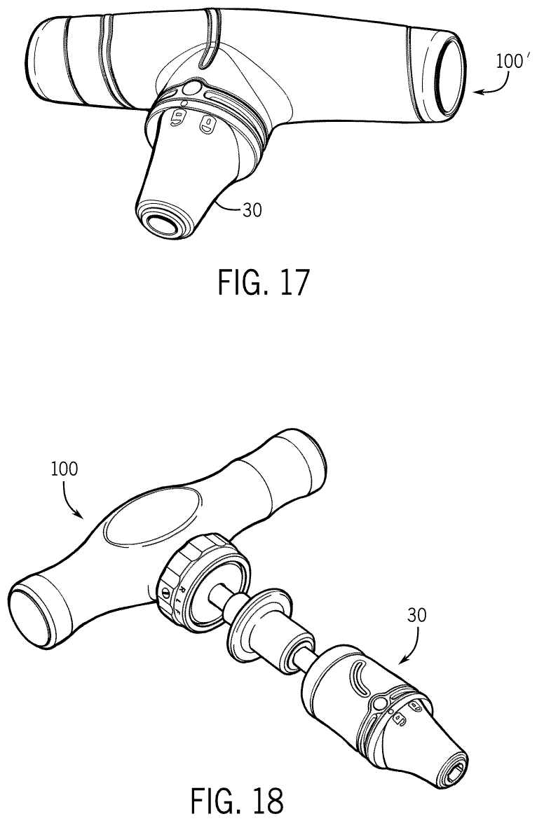

Looking now at FIGS. 15-18, the adapter 30 can be formed for use with an in-line tool 102 (FIG. 16) or a T-handle tool 100 (FIG. 18) in which the shaft 22 is replaced by a second sleeve (not shown) adapted to receive the output shaft (not shown) of the in-line tool 102 or the T-handle tool 100, or as an integral part of an in-line device 102′ (FIG. 15) or a T-handle device 100′ (FIG. 17), where the adapter 30 is configured as a single use adapter 30 that is optionally disposable.

Referring now to the exemplary embodiments of FIGS. 6, 7, and 11, the adaptor 30 can be releasably connected to the powered driving tool 10 between the driving tool 10 and the driven implement 40, such as a drill or saw or some other powered implement, in order to provide the motive power for the driven implement 40, such as by rotating the driven implement 40 via the rotation of the shaft 22 in the adapter 30 powered by the driving tool 10. The adaptor 30 can be releasably connected between the driving tool 10 and the driven implement 40, such by employing the cover 45 and gripping ring 300, and/or by other suitable mechanisms, e.g., a chuck mechanism, to form a combined instrument system. In this configuration, with the information stored on the adapter 30 and/or communicated to the adapter 30 via the remote platform 508, such that the adapter 30 can provide an alarm and/or disconnect the driven implement 40 from the driving tool 10 if certain programmed criteria, e.g., a maximum torque value, were exceeded.

In another embodiment, where one of the driving tool 10 or the adapter 30 includes the electromagnetic clutch mechanism 74, the electromagnetic clutch mechanism can 74 be operated to disengage and stop the driven implement 40 if certain forces and/or signals sensed by the adapter 30, e.g., torque or vibration, were to go outside criteria programmed into the adapter 30 and/or remote platform 508.

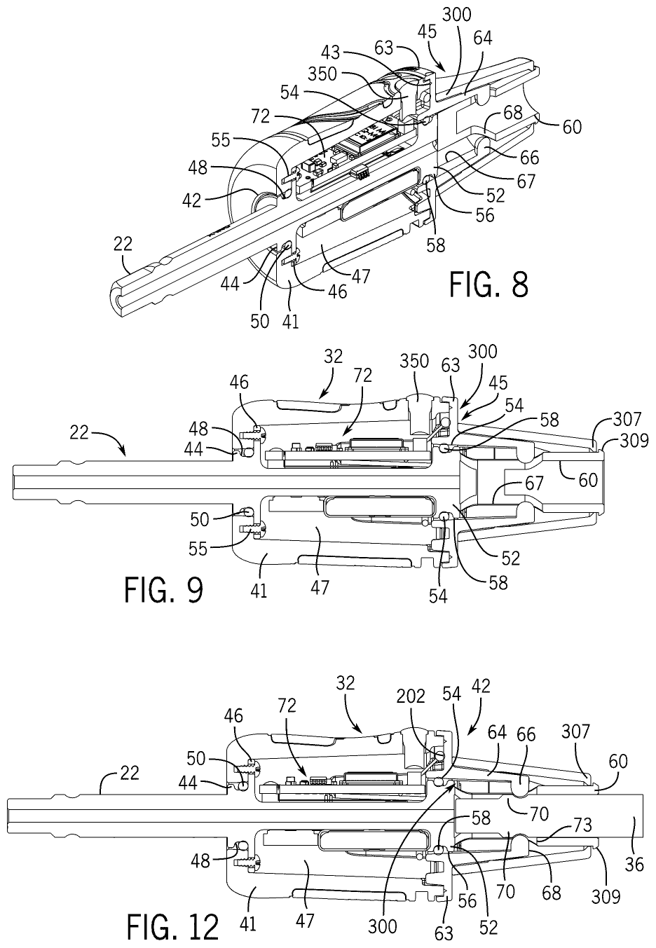

In any configuration, the adapter 30 can also include one or more indicators 350 (FIGS. 7 and 8) operably connected to the PCB/electronics/control unit 72 that can indicate power being supplied to the adapter 30 and/or any of a number of states of operation of the adapter 30/tool 10 using visual, audible and/or tactile indications provided by the one or more indicators 350, such as any alarm condition for the operation of the adapter 30/tool 10.

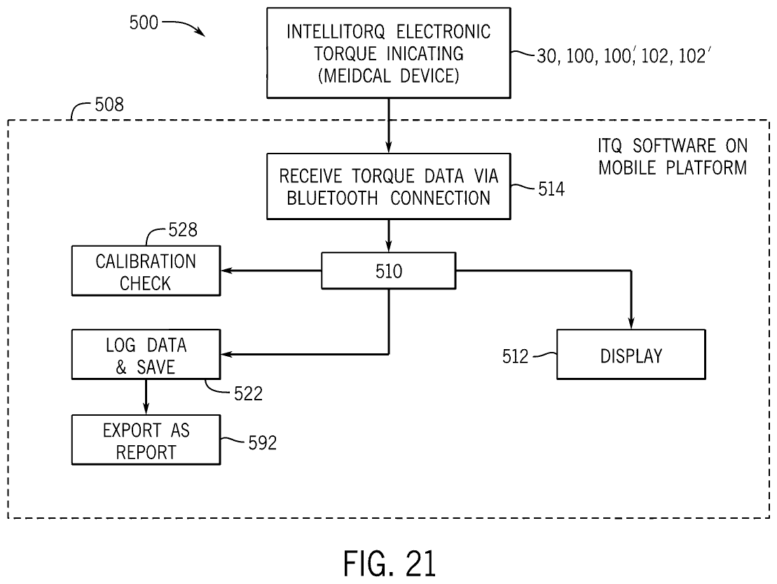

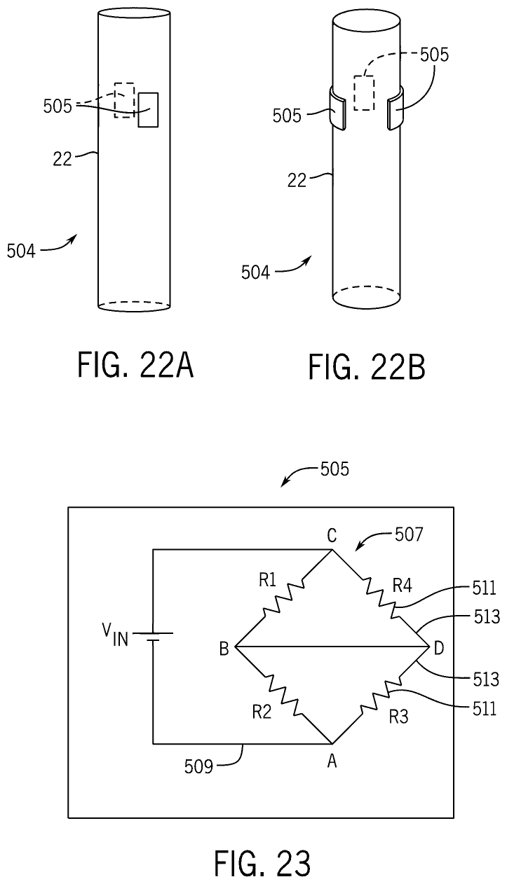

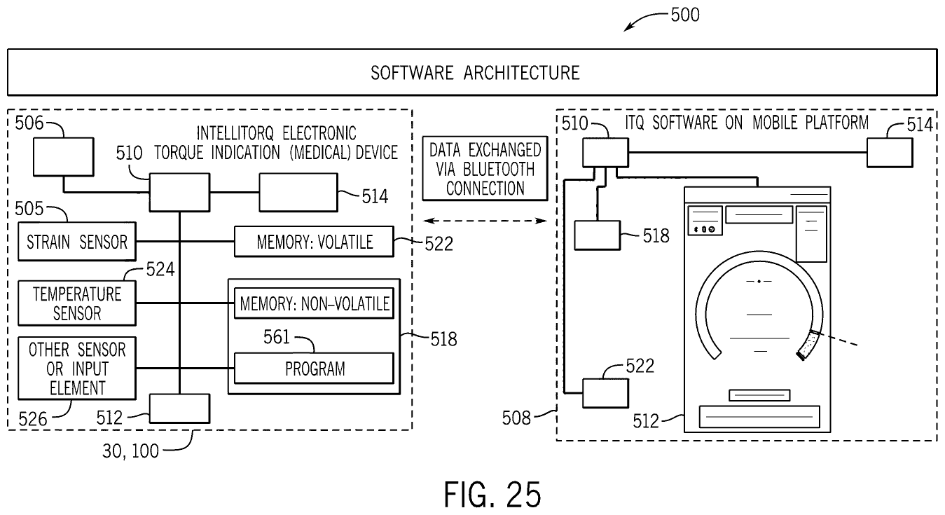

In addition, either in conjunction with the adapter 30 and/or with another torque measurement device or tool 10, 100, 102, 102′, whether including the torque detection capability integrally within the device or tool 10, 100, 102, 102′ or by using the adapter 30, as described previously, the tool 10, 100, 100′, 102, 102′ and/or the adapter 30 can be employed as a part of a self-check and torque measurement and analysis system 500, illustrated schematically in FIGS. 21-23. The self-check and torque measurement and analysis system 500 includes the tool or device 10, 100, 102, 102′ or adapter 30 having a torque measurement device 504 incorporated therein and operably connected to the PCB/electronics/control unit 72. As shown in FIGS. 22A-23, the torque measurement device 504 includes at least one torque measurement sensor 505 located on the shaft 22, such as a strain gauge 507 that in one exemplary embodiment is formed of a Wheatstone bridge circuit 509 including a number of resistors 511 disposed along arms 513 for the circuit 509. The output Vₒᵤₜ of the circuit 509 can be determined or the read for changes in, i.e., strain determined by, any one or more of the components of the one or more sensors 505, e.g., the resistors 511, to provide torque data to the PCB/electronics/control unit 72 during the operation or use of the tool 10, 100, 100′, 102, 102′ and/or the adapter 30. The PCB/electronics/control unit 72 can store the torque measurement data in associated memory (522, FIG. 25) within the tool 10, 100, 102, 102′ and/or the adapter 30 and/or can additionally include a wireless transmitter or transceiver 506 capable of transmitting torque data obtained during use of the torque measurement device 504 to a remote receiver (to be described), along with other optional features, such as a display (not shown) and a haptic feedback device (not shown).

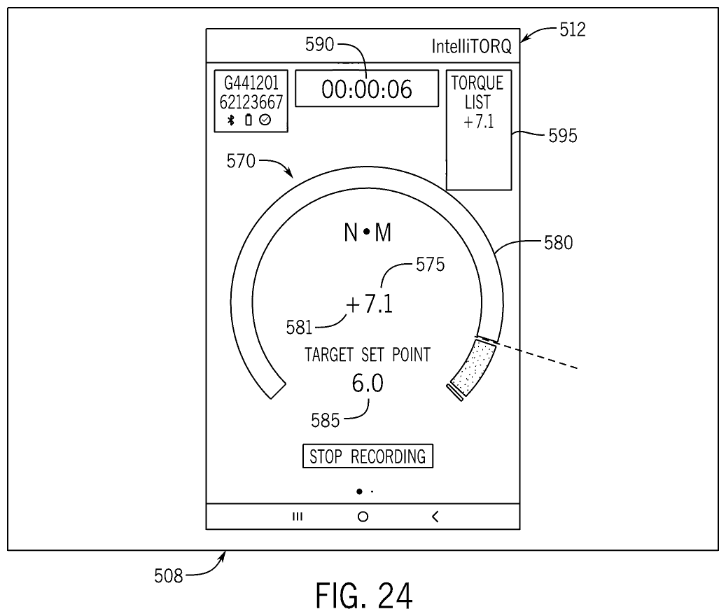

Referring now to FIGS. 24-25, with the wireless transmitter or transceiver 506 the torque data is transmitted from the PCB/electronics/control unit 72 to a remote platform 508, which in one embodiment can take the form of a computer, smartphone or tablet computer 508′ including a central processing unit (CPU) 510, among other suitable devices. One or both of the tool 10, 100, 102, 102′ and/or the adapter 30 and/or the remote platform 508 includes a display 512 and a transceiver 514 for sending information to and receiving information from the tool 10, 100, 102, 102′ and/or the adapter 30, over either a wired or wireless, e.g., Bluetooth® connection, including the torque data obtained by the tool 10, 100, 102, 102′ and/or the adapter 30 using the torque measurement device 504 from the at least one component of the at least one sensor 505. The remote platform 508 can present the torque data in real-time on the display 512 along with other relevant information stored in the electronic memory 518 of the remote platform 508 concerning the use of the tool 10, 100, 102, 102′ and/or the adapter 30, including, but not limited to information regarding the tool 10, 100, 102, 102′ and/or the adapter 30, e.g., type, manufacturer, etc., the selected torque units, the direction of the application of the torque, a target torque set point, a list of peak torque values applied during the use of the tool 10, 100, 102, 102′ and/or the adapter 30, the date, time and duration of the session of use of the tool 10, 100, 102, 102′ and/or the adapter 30 potentially along with data on the patient with whom the tool 10, 100, 102, 102′ and/or the adapter 30 was utilized, the real-time torque value (which can be numerically and/or graphically illustrated in one or more colors), and one or more control function button for the system 500, such as a start/stop button 516 for recording the torque data from the tool 10, 100, 102, 102′ and/or the adapter 30.

In an alternative embodiment, either separately from or in conjunction with the above information, the torque data can be presented in the form of a graph or track of the applied torque over the course of the session of use of the tool 10, 100, 102, 102′ and/or the adapter 30.

Further, the remote platform 508 includes non-volatile electronic memory 518 that is operably connected to an internal computer or central processing unit (CPU) 520 that accesses computer-executable operational instructions and/or algorithms 561 within the memory 518 to operate the torque measurement and analysis system 500 and provide the torque data representations on the display 512. The remote platform 508 additionally includes volatile electronic memory 522 that stores the torque data received from the tool 10, 100, 102, 102′ and/or the adapter 30 during the operation of the tool 10, 100, 102, 102′ and/or the adapter 30. The torque data stored in volatile memory 522 can be employed by the processing unit (CPU) 520 using the information and instructions stored in the non-volatile memory 518 to provide the information on the display 512 and to provide transmittable/exportable reports or data files for individual sessions of use of the tool 10, 100, 102, 102′ and/or the adapter 30.

In an alternative embodiment of the torque measurement and analysis system 500 illustrated in FIG. 25, one or more of the components of the remote platform 508, e.g., the non-volatile memory 518 and volatile memory 522, can be located on the tool 10, 100, 102, 102′ and/or the adapter 30 in operable connection to the PCB/electronics/control unit 72.

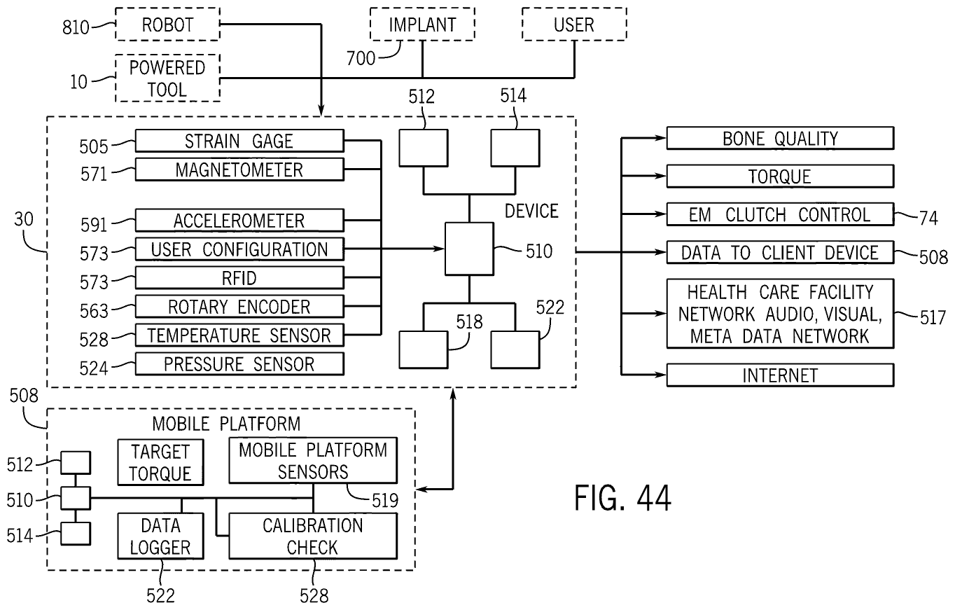

In addition, with reference to FIG. 44, the torque measurement device 504 can optionally include a temperature sensor 524 and an user configuration/input device 526 (which can alternatively be operably connected to the electronic unit 72) such as a push button array to enable the user to input information directly into the tool 10, 100, 102, 102′ and/or the adapter 30. Further, the tool 10, 100, 102, 102′ and/or the adapter 30 can be configured to include as part of the torque measurement device 504 one or more of the torque/strain sensor/strain gauge 505, a magnetometer 571, an accelerometer 591, a radio frequency identification (RFID) scanner 573 for identifying fasteners 700 and other components utilized with the tool 10, 100, 102, 102′ and/or the adapter 30, and a rotary encoder 563 to measure and record the rotation of the shaft 22, each of which are operably connected to the PBC/electronics/control unit 72.

With one or more of these features on the tool 10, 100, 102, 102′ and/or the adapter 30, which can be attached to a robotic arm 811, output from the PBC/electronics/control unit 72 can include evaluations of the bone quality into which the tool 10, 100, 102, 102′ and/or the adapter 30 is driving the fastener 700, the torque applied to drive the fastener, any clutch control applied to the shaft 22 using the clutch mechanism 74, and the data regarding these measured values sent to the remote device 508, another computer network 517, or transmitted via the internet.

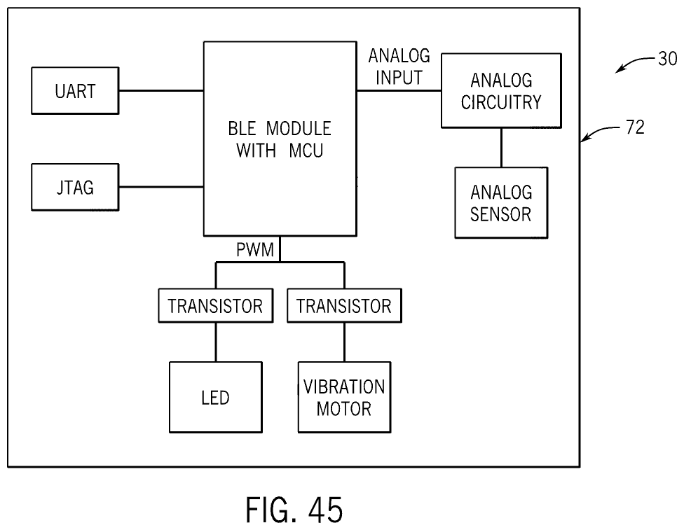

Further, the tool 10, 100, 102, 102′ and/or the adapter 30 and/or the remote platform 508 can include a calibration program 528 used to provide a calibration self-check for each of the tool 10, 100, 102, 102′ and/or the adapter 30 and/or the remote platform 508 in manner to be described. Further, the remote platform 508 can include additional sensors 519, a data logger/volatile electronic memory 522 for recording data from the various sensors 505,507,509,511, and non-volatile electronic memory 518 for storing information concerning various medical procedures, fastener locations and associated maximum torque values. Alternative exemplary embodiments of the configuration for the operation and interaction of the system 500 and mobile and/or remote platform 508 with the adapter 30 and/or tool 10, 100, 102, 102′ and for the hardware for the system 500 and/or mobile or remote platform 508 are shown schematically in FIGS. 44 and 45.

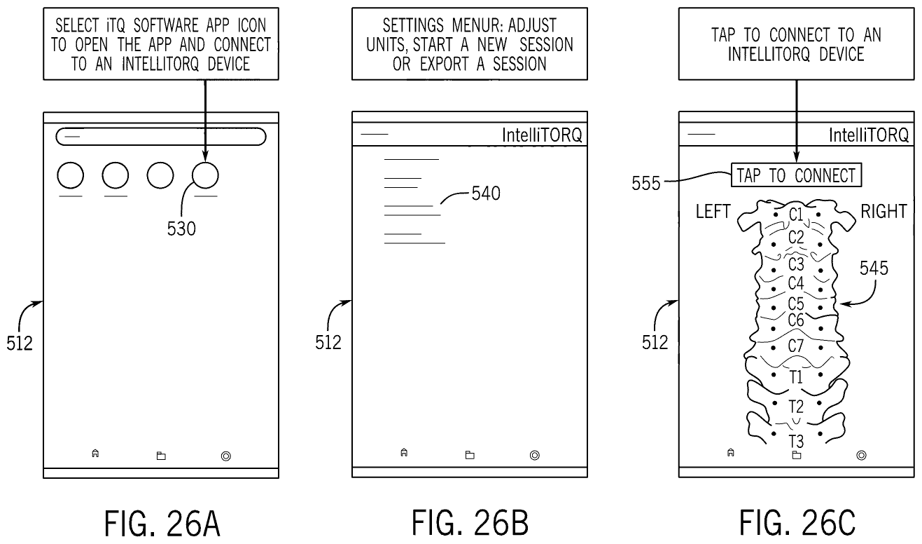

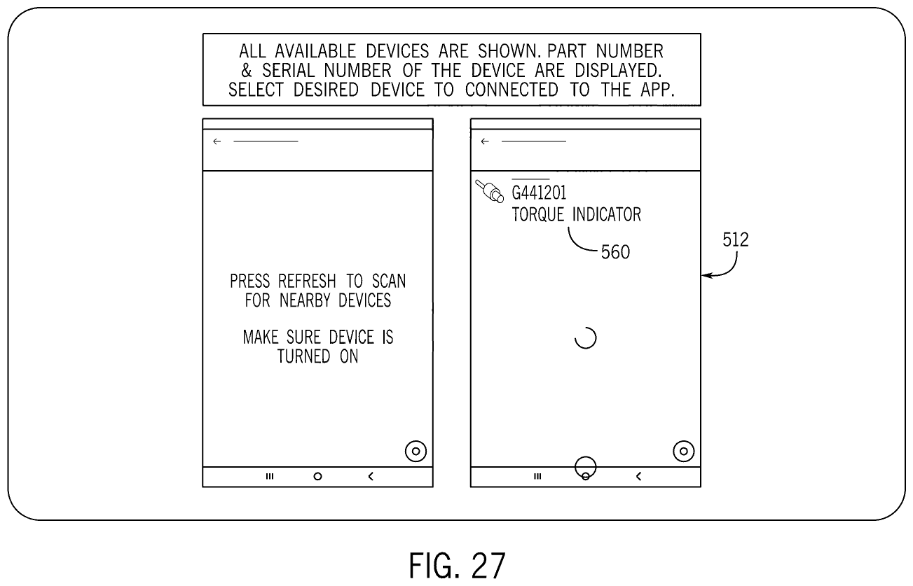

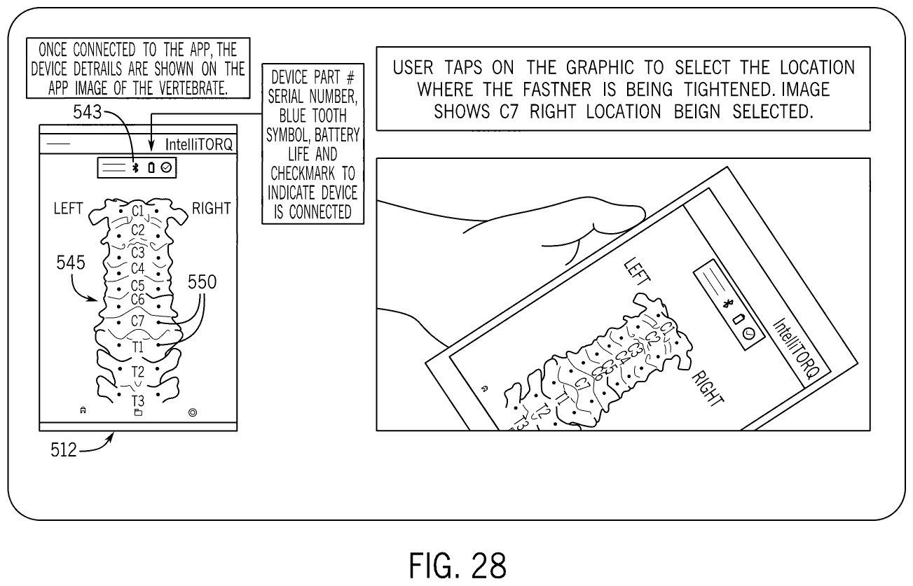

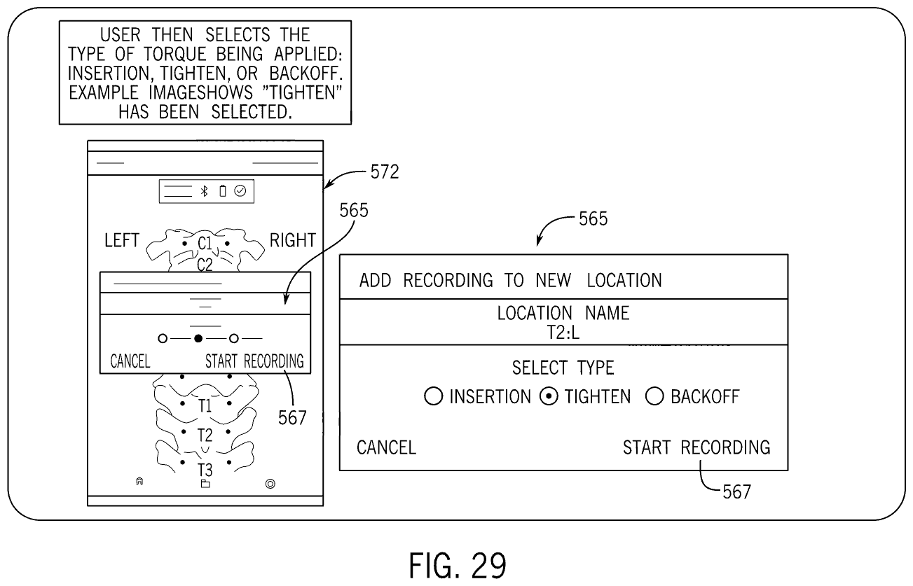

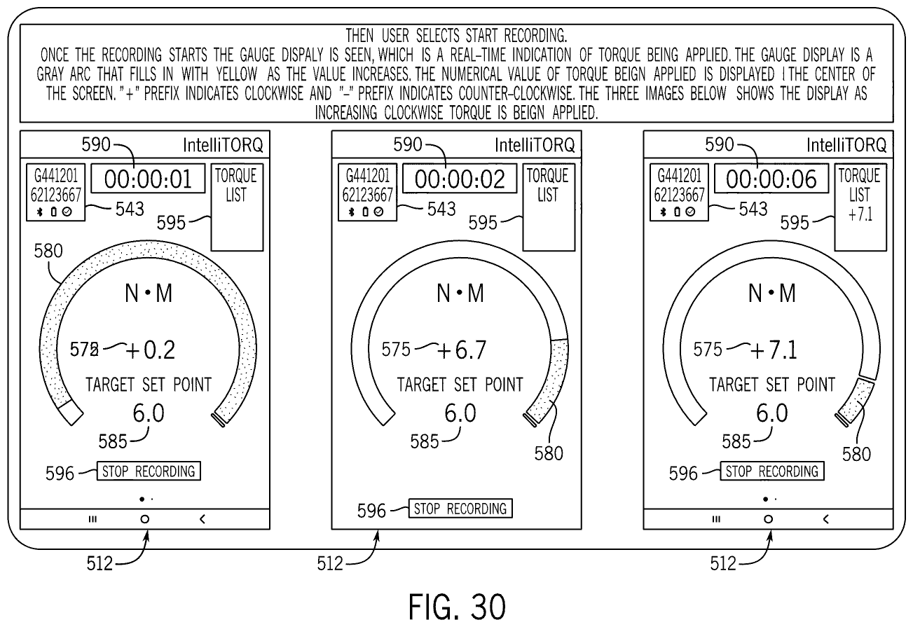

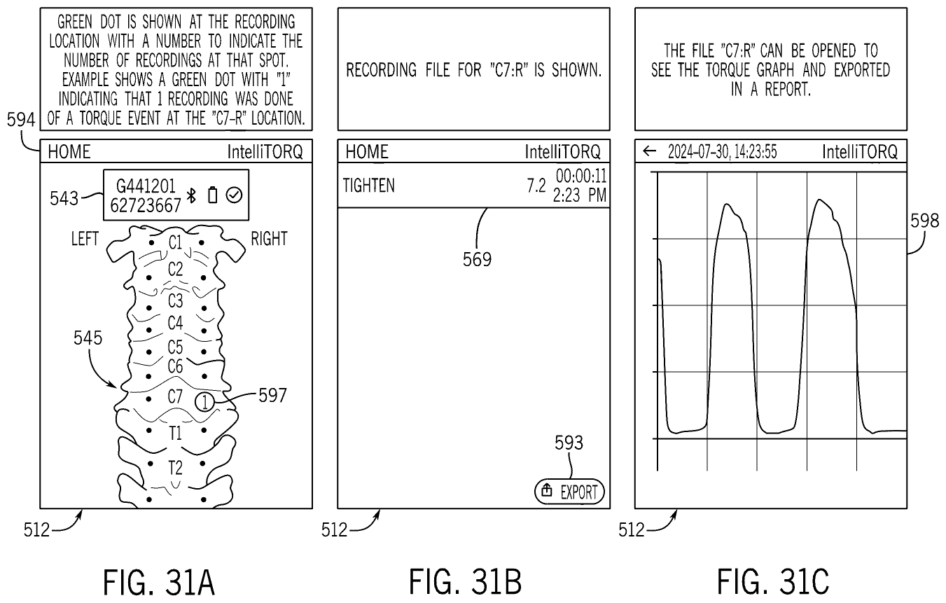

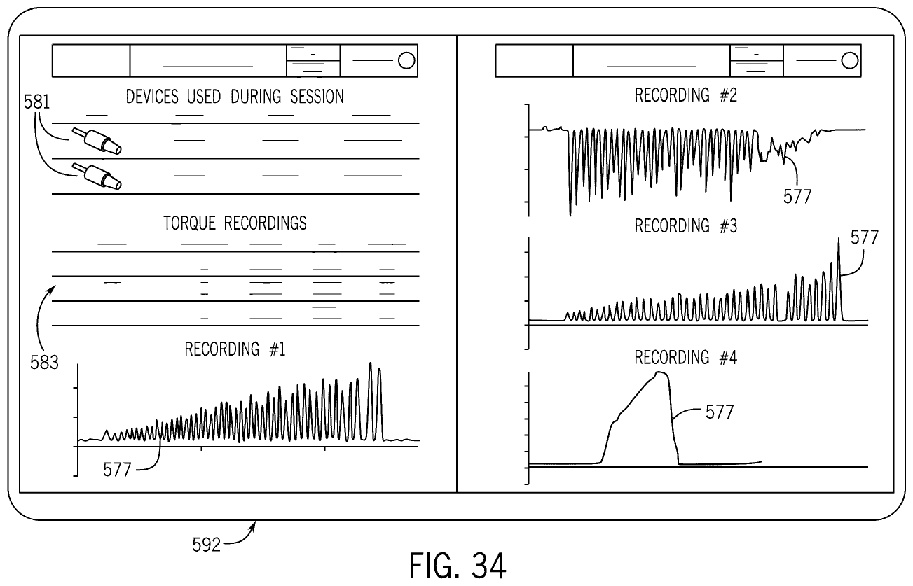

Looking now FIGS. 26A-34, an exemplary embodiment of the method of operation of the self-check and torque measurement and analysis system 500 is illustrated following a general flow of the steps for one example of the use of the self-check and torque measurement and analysis system 500 during a medical procedure, e.g., a spinal procedure.
1. Select the icon 530 on the display 512 of the home screen 535 of the remote device 508, e.g., the tablet computing device or smartphone, to execute the stored instructions for the operation of the self-check and torque measurement system 500 contained on the remote device 508 (FIG. 26A).
2. Upon execution settings page 540 initially presented to adjust units for measurement recording, to export stored session/procedure or to start new session/procedure (FIG. 26B).
3. When selecting new procedure, menu provided where the specific procedure can be selected, which can be any suitable procedure, such as a medical or dental procedure, where screws and/or other types fasteners are to be inserted using the tool 10 and/or the adapter 30 including the toque measurement device 504—for selection of the procedure a library of procedures, the associated anatomical structures and the locations and numbers of fasteners or screws can be stored in nonvolatile memory 518 on the remote platform 508 and accessed when the application for the system 500 is activated (information on each procedure can include information relating to various types of fasteners, screws, and other components, including saddles, tulips and/or bars, to be used in the procedure that have various torque values associated with the proper insertion of the components supplied from the respective manufacturers of the fasteners.
4. Upon selecting particular procedure for new session, system 500 accesses stored information on the selected procedure (e.g., spinal procedure) and presents on the display 512 is a line image of the anatomical structure 545 associated with the selected procedure e.g., a dorsal view of spinal column, with dots or other indications 550 marked on each the anticipated fastener, e.g., pedicle screw, attachment or insertion point. (FIG. 26C)
5. If improper procedure/anatomical structure 545 selected and presented, can return to previous selection menu. Alternatively, if proper anatomical structure 545 for selected procedure presented on display 512, can select ‘Tap To Connect’ button 555 to initiate wireless connection of remote device 508 via transceiver 514 to compatible tool 10, 100, 102, 102′ and/or adapter 30 (FIG. 26C)
5. Remote platform 508 performs scan via Bluetooth or other suitable wireless connection protocol for available tool 10, 100, 102, 102′ and/or adapter 30 and presents icon(s) 560 representing each active (powered on) compatible tool 10, 100, 102, 102′ and/or adapter 30 in range on the display 512, such that user can select icon 560 to connect to desired tool 10, 100, 102, 102′ and/or adapter 30. (FIG. 27)
6. When a connection made between remote platform 508 and desired tool 10, 100, 102, 102′ and/or adapter 30, system 500 presents on display 512 view of selected anatomical structure 545 for procedure with status 543 (e.g., charge level) and other information regarding the tool 10, 100, 102, 102′ and/or adapter 30 above the anatomical structure 545. (FIG. 28).
7. On the anatomical structure 545 presented on the display 512 a target location/dot 550 is identified and a menu 565 is displayed for selection of type of torque to be applied for that location (insertion, tighten or backout—these different types of torque can be required for different components, e.g., insertion screws and set screws, to be utilized in the procedure and/or for adjustment of initial torque to place fastener within desired torque range for fastener and/or procedure) (FIG. 29).
8. Select ‘Start Recording’ 567 on menu 565 to begin recording of torque data generated by the torque measurement device 504, the tool 10, 100, 100′, 102, 102′ and/or adapter 30 for transmission to the remote platform 508 when engaging and rotating fastener (FIG. 29).
9. After beginning recording, system 500 changes display 512 to gauge view 570 which shows real-time +/-value of torque being applied 575, in conjunction with color coded dial/arc of application torque range 580, torque set point 585 numerically and on arc 580, elapsed time for torque application 590, and highest torque value applied 595. Values are provided numerically with units (N.m=Newton Meter) and direction of torque 581 ‘+’ (+=Clockwise) (FIG. 30).
10. After tightening of the fastener with the tool 10, 100, 102, 102′ and/or adapter 30 is finished, the session is complete, and the ‘Stop Recording’ button 596 on the display 512 is selected to stop the recording of the torque data from the tool 10, 100, 102, 102′ and/or adapter 30. (FIG. 30)
11. When the torque data recording stops, the system 500 presents on the display 512 the anatomical structure 545 (e.g., spine) with green dot/recording icon 597 to illustrate the location on the anatomical structure 545 where the fastener was inserted and the recording was done. (FIG. 31A)
12. The torque data recorded in real-time at each individual location 560 is stored, e.g., in volatile memory 522 of the remote platform 508 or in any other suitable electronic storage location in association with the procedure. During or after a procedure has been completed, the recorded torque data can be accessed by selecting the recording icon 597 to present a summary 569 of the recorded torque data (FIG. 31B) and/or a graphical representation 598 of the application of the recorded torque (FIG. 31C).
13. From the presentation of the recorded torque data for a performed procedure, i.e., the anatomical structure 545 showing each of the recording icons 597 for the locations 560, the user can return to the setting screen (FIG. 26B) with the Home button 594 (FIG. 31A), or can select the Export button 593 to send a preformatted report 592 (FIG. 34) of the selected recording icon 597 or the entire recorded procedure. (FIG. 31B).

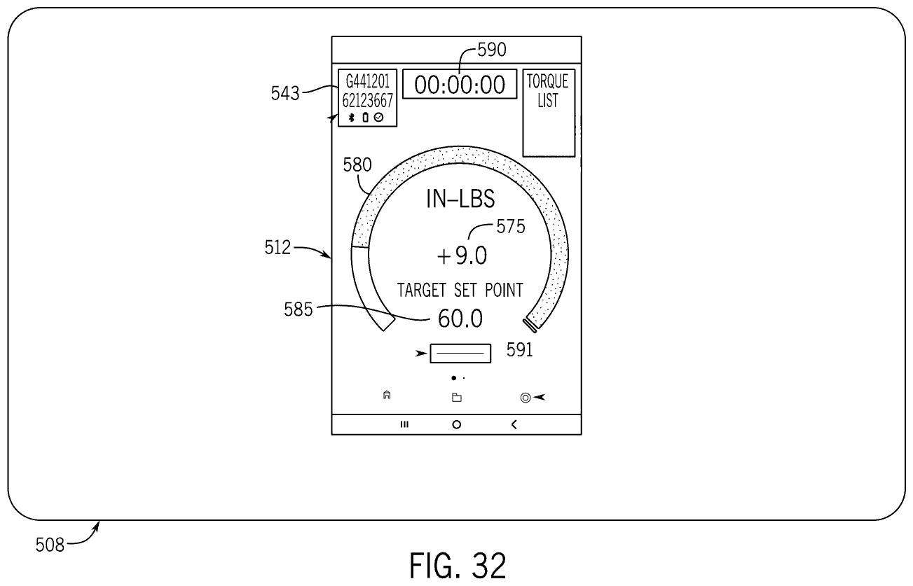

In an alternative embodiment shown in FIG. 32, the torque data can be selected to be presented on the display 512 in real-time in the form of a graph or track (FIG. 31C) of the applied torque over the course of the session of use of the tool 10, 100, 102, 102′ and/or adapter 30 as opposed to a dial/arc showing the current applied torque value as a colored bar travelling along the dial in response to the increase or decrease of the torque applied by the tool 10, 100, 102, 102′ and/or adapter 30. Additionally, the system 500 can enable the target torque to be modified by the user on the screen 512 with a selectable Set Target Torque button 591 to provide a calibration check functionality for the system 500.

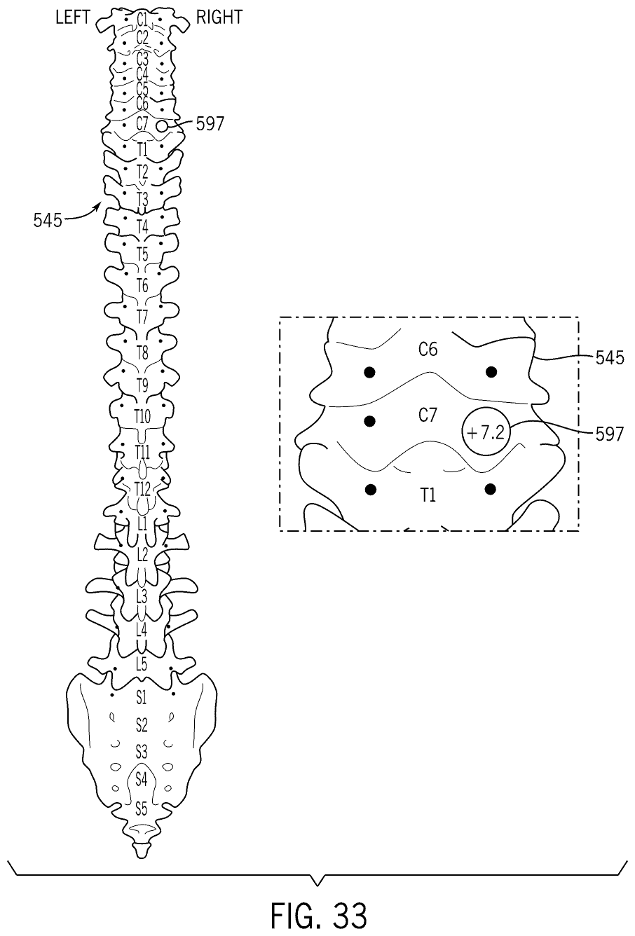

In still another alternative embodiment shown in FIG. 33, the dots/recording icons 597 illustrated on the anatomical structure 545 for a procedure representing fasteners that have already been completed are shown on the anatomical structure with values of the maximum applied torque illustrated within the recording icons 597.

With regard to FIG. 34, an exemplary embodiment a report 592 output by the remote platform 508 for each of the recordings done during a procedure is illustrated as presented on a display 512, such as the recordings of the tightening of each fastener. The report 592 includes a list of the tool 10, 100, 102, 102′ and/or adapter 30 utilized in the procedure 581 and a log 582 for the procedure showing six recorded torque events 583, including the tool 10, 100, 102, 102′ and/or adapter 30 used for each, the type of event and the max torque applied. Serial numbers of the tool 10, 100, 102, 102′ and/or adapter 30 used can also be noted in the report 592. Each recording can also be graphically illustrated with a track or graph 577 tracing each torque value recorded in the particular event and illustrating the click-overs also performed by the tool 10, 100, 102, 102′ and/or adapter 30 during the recorded event.

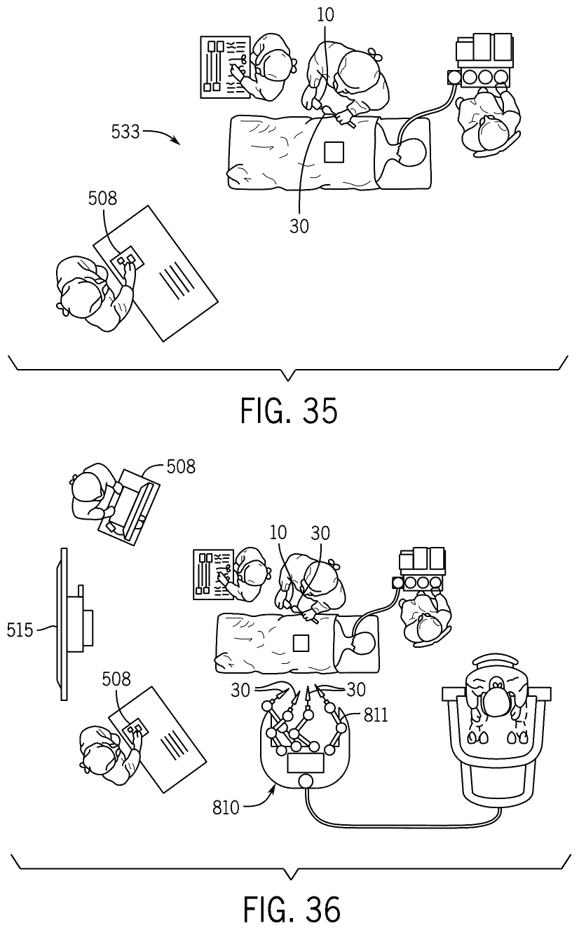

Turing now to FIG. 35, in an exemplary embodiment for the use of the system 500, the tool 10 and/or adapter 30 is employed by a physician in an operating theater 533 to perform the insertion of the fasteners or screws for the particular procedure. The remote platform 508 is held by a second individual outside of the sterile area/field, such as a circulating nurse, that operates the remote platform 508 to record the various torque events performed by the physician during the procedure. In the exemplary embodiment of FIG. 36, the system 500 and tool 10 and/or adapter 30 is utilized in an operating theater including a monitor 515 operably connected to the tool 10, adapter 30 and or remote platform 508 to function as the display 512 and a medical robot 810 operated remotely by the physician to perform the operation, where the adapter 30 and/or the tool 10, 100, 102, 102′ is employed on one or more arms 811 of the robot 810.

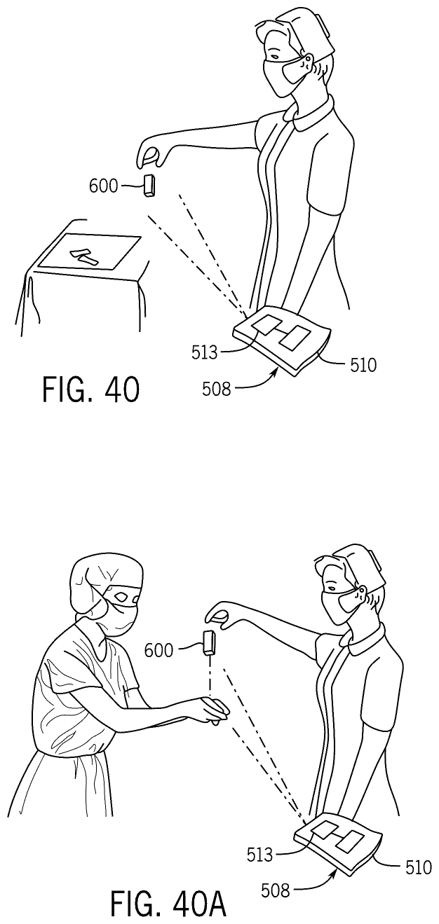

In FIGS. 40-40A, tags 600 associated with the various implements 36 and/or components 700 used in a procedure conducted using the tool 10 and adapter 30 along with the system 500. The tags 600 can be scanned via the RFID scanner 573 to record the identifying information on the components 700 (e.g., manufacturer, type, batch number, etc.) within the record for the procedure along with the stored torque data. The tags 600 can be dropped outside of the sterile zone in the operating theater into a non-sterile area in order to maintain sterility within the sterile zone where the procedure is being performed.

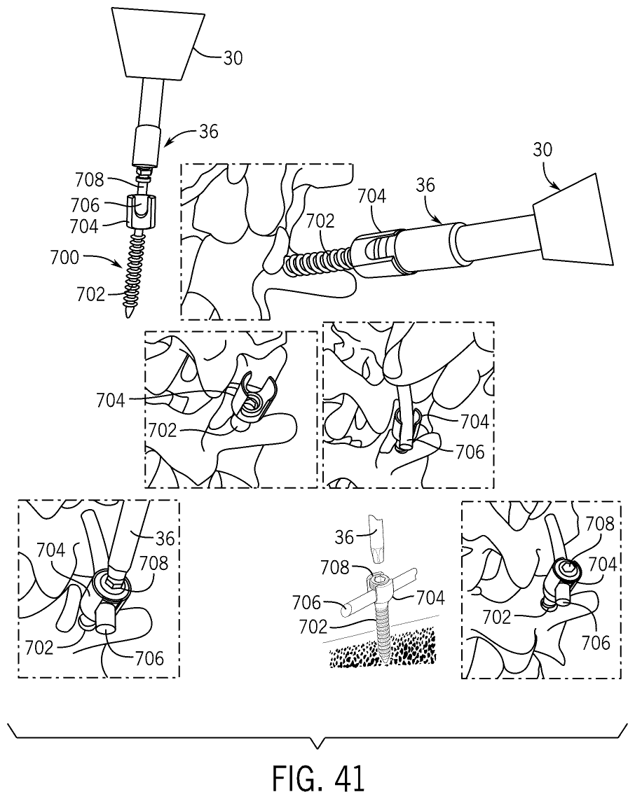

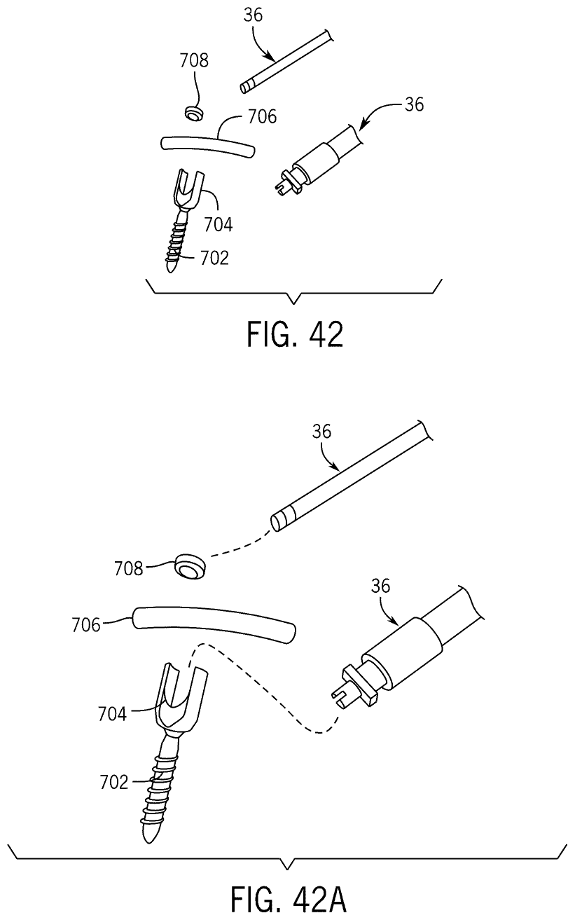

In FIGS. 41-42, the steps and components 700 for performing a spinal procedure with the adapter 30, tool 10 and system 500 are illustrated in an exemplary embodiment of the disclosure showing the insertion of a fastener/component 700 into a spine, such as in a medical procedure illustrated in FIGS. 26-34. The components 700 can include a pedicle screw 702 with a tulip 704 at one end, a rod 706 and a set screw 708, where the screw 702, tulip 704 and set screw 708 can be engaged with an implement 36 secured to the adapter 30 and tool 10.

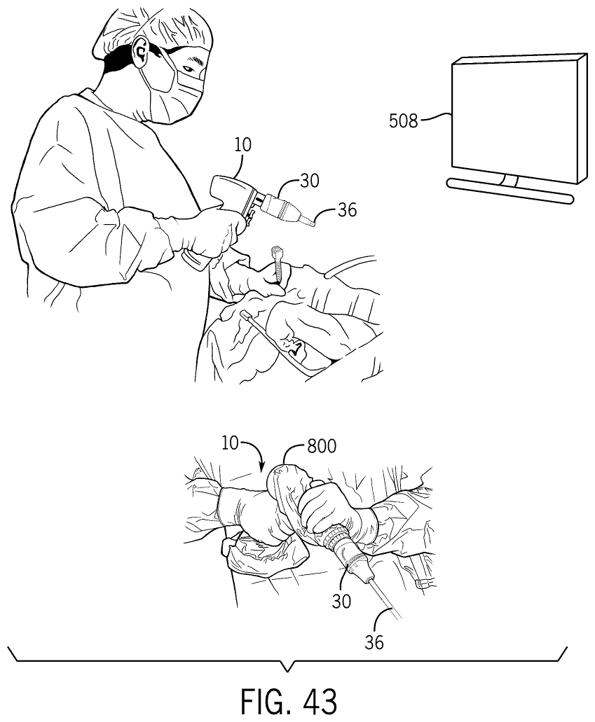

Finally in FIG. 43, the tool 10 and adapter 30 are illustrated in use in both a sterile configuration (no drape around tool 10) and an unsterile configuration (a drape 800 positioned around the tool 10).

In addition to the stored recordings being utilized to generate reports 592 of the torque events for a procedure, the recorded torque data can be employed by the tool 10, 100, 102, 102′ and/or adapter 30 and/or the system 500 on the remote platform 508 to provide a calibration or self-check of the tool 10, 100, 102, 102′ and/or adapter 30 and of the remote platform 508. Alternatively, or in addition to the internal calibration, the tool 10, 100, 102, 102′ and/or adapter 30 can be engaged with a suitable external calibration device or mechanism to provide the calibration and verification of the accuracy of the torque measurements provided by the tool 10, 100, 102, 102′ and/or adapter 30.

While the concepts of the present disclosure will be illustrated and described in detail in the drawings and description, such an illustration and description is to be considered as exemplary and not restrictive in character, it being understood that only the illustrative embodiments are shown and described and that all changes and modifications that come within the spirit of the disclosure are desired to be protected. There are a plurality of advantages that may be inferred from the present disclosure arising from the various features of the apparatus, systems, and methods described herein. It will be noted that alternative embodiments of each of the apparatus, systems, and methods of the present disclosure may not include all of the features described yet still benefit from at least some of the inferred advantages of such features disclosed in other embodiments which are deemed to be included in the disclosures of each of the various embodiments disclosed herein as well as in the disclosures of U.S. Pat. Nos. 8,485,075; 8,714,058; 9,358,672; 9,505,109; 10,046,445 and 10,987,785, each of which are expressly incorporated herein by reference in their entirety for all purposes. Those of ordinary skill in the art may readily devise their own implementations of an apparatus, system, and method that incorporate one or more of the features of the present disclosure and fall within the spirit and scope of the disclosure as defined by the appended claims.

We claim:

1. A torque measuring adapter for a driving tool, the adapter comprising:
a) a body;
b) a shaft extending upwardly from the body and adapted to be engaged with a driving tool; and
c) an electronics unit including a torque measurement system operably connected to the shaft and operable to measure the torque exerted though the shaft on a fastener.

2. The torque measuring adapter of claim 1, wherein the body comprises:
a) a housing enclosing the electronics unit, the torque measurement system and the shaft;
b) a sleeve engaged with the shaft and extending through the cover, the sleeve adapted to engage an implement therein; and
c) a cover rotatably secured to housing around the sleeve and opposite the shaft.

3. The torque measuring adapter of claim 2, further comprising a gripping ring secured to the housing within an interior of the cover, the gripping ring including a base secured to the housing and a pair of arms extending outwardly from the base and including tabs disposed opposite the base, the tabs adapted to selectively extend into the sleeve and grip an implement positioned within the sleeve.

4. The torque measuring adapter of claim 3 wherein the cover is rotatable with regard to the gripping ring, and wherein the cover includes a number of engaging surfaces thereon that are engageable with the arms upon rotation of the cover to selectively move the tabs into and out of the sleeve.

5. The torque measuring adapter of claim 2, further comprising a power switch disposed in the housing that is operably connected to the electronics unit and selectively activated by rotation of the cover relative to the housing.

6. The torque measuring adapter of claim 5, wherein the cover includes at least one magnet disposed therein, where the cover is rotatable to selectively position the magnet in alignment with the power switch to activate the power switch.

7. The torque measuring adapter of claim 6 wherein the cover includes a pair of magnets spaced from one another and selectively alignable with the power switch.

8. The torque measuring adapter of claim 7, wherein the cover is rotatable relative to the housing between:
a) a first position where the power switch is deactivated and no implement is engaged within the sleeve;
b) a second position where the power switch is activated and no implement is engaged within the sleeve; and
c) a third position where the power switch is activated and an implement is engaged within the sleeve.

9. The torque measuring adapter of claim 5, wherein the power switch is a reed switch.

10. The torque measuring adapter of claim 1, wherein the torque measurement system includes at least one torque measurement sensor disposed on the shaft and operably connected to the electronic unit.

11. The torque measuring adapter of clam 10, wherein the torque measuring system further comprises:
a) a first transceiver operably connected to the electronics unit within the adapter for transmitting torque data obtained from the at least one torque measurement sensor; and
b) a remote platform including:
i) a second transceiver for receiving the torque data from the torque measurement system;
ii) a display;
iii) a central processing unit (CPU) configured to process the torque data for presentation on the display; and
iv) electronic memory including CPU-executable operational instructions for operating the torque measuring system, and analyzing and storing the torque data.

12. The torque measuring adapter of claim 11, wherein the remote platform is formed as part of the torque measuring adapter.

13. The torque measuring adapter of claim 1, wherein the adapter is attached between a driving tool and a driven implement.

14. The torque measuring adapter of claim 13, wherein the adapter further comprises a clutch mechanism operably engaged between the driving tool and the driven implement.

15. The torque measuring adapter of claim 1, wherein the adapter is attached to an arm of a surgical robot.

16. A torque measurement and analysis system for recording applied torque measurement data during operation of a driving tool in a procedure, the system comprising:
a) torque measuring adapter connected to a driving tool, the adapter comprising:
i) a housing;
ii) a shaft extending outwardly from one end of the housing and adapted to be engaged with a driving tool
iii) a sleeve engaged with the shaft and extending outwardly from the other end of the housing, the sleeve adapted to engage an implement therein;
iv) a cover rotatably secured to the housing around the sleeve and opposite the shaft;
v) an electronics unit disposed within the housing and operably connected to the shaft, the electronics unit including:
a) a torque measurement sensor operably connected to the shaft and operable to measure the torque exerted though the shaft on a fastener to generate torque data; and
b) a first transceiver for transmitting the torque data from the torque measurement sensor; and
b) a remote platform including:
i) a second transceiver for receiving the torque data from the electronics unit;
ii) a display;
iii) a central processing unit (CPU) configured to process the torque data for presentation on the display; and
iv) electronic memory including CPU-executable operational instructions for operating the torque measuring system, and analyzing and storing the torque data.

17. The torque measurement and analysis system of claim 16, wherein the electronic memory stores information regarding various procedures and to be performed with the torque measuring adapter, and wherein the CPU is configured to:
a) present an anatomical structure associated with a selected procedure on the display; and
b) present locations for insertion of fasteners using the torque measuring adapter in association with the anatomical structure.

18. The torque measurement and analysis system of claim 17, wherein the CPU is configured to:
a) acknowledge a selected fastener location on the anatomical structure;
b) receive torque data from the torque measuring adapter regarding the insertion of a fastener at the selected fastener location; and
c) display the recorded torque data.

19. The torque measurement and analysis system of claim 18, wherein the CPU is configured to:
a) present a current applied torque value from the torque measuring adapter;
b) present a maximum applied torque value from the torque measuring adapter; and
c) present a torque set point for the selected fastener location.

20. The torque measurement and analysis system of claim 18, wherein the CPU is configured to:
a) record torque data from the insertion of a fastener at the selected fastener location; and
b) present a report with the recorded torque data from the insertion of a fastener at the selected fastener location.
